(19) United States
(12) Patent Application Publication
(10) Pub. No.: US 2023/0137729 A1
(43) Pub. Date: May 4, 2023

(54) METHODS, COMPOSITIONS AND COMPONENTS FOR CRISPR-CAS9 EDITING OF CBLB IN T CELLS FOR IMMUNOTHERAPY

(71) Applicants: EDITAS MEDICINE, INC., Cambridge, MA (US); JUNO THERAPEUTICS, INC., Seattle, WA (US)

(72) Inventors: Andreia S. COSTA, Seattle, WA (US); Jason David FONTENOT, Seattle, WA (US); Fred HARBINSKI, Cambridge, MA (US); Gordon Grant WELSTEAD, Cambridge, MA (US); Chris WILSON, Cambridge, MA (US)

(21) Appl. No.: 16/758,843

(22) PCT Filed: Nov. 5, 2018
(86) PCT No.: PCT/US2018/059146

§ 371 Date: Apr. 23, 2020

Related U.S. Application Data
(60) Provisional application No. 62/582,393, filed on Nov. 7, 2017.
(60) Provisional application No. 62/582,020, filed on Nov. 6, 2017.

(51) Int. Cl. C12N 15/113; C12N 9/22

(52) U.S. Cl. CPC C12N 15/1135; C12N 15/1138; C12N 9/22; C12N 2310/20

(57) ABSTRACT

CRISPR/CAS-related genome editing systems, compositions and methods for targeting the CBLB locus, as well as cells edited using these systems, compositions and methods are provided.

### RELATED APPLICATIONS

This application is a 35 U.S.C. § 371 filing of International Patent Application No. PCT/US2018/059146, filed Nov. 5, 2018, which claims the benefit of U.S. Provisional Application Ser. Nos. 62/582,020, filed on Nov. 6, 2017, and 62/582,393, filed on Nov. 7, 2017, each of which is incorporated herein by reference in its entirety.

### FIELD

The present disclosure relates to CRISPR/Cas9-related methods and components for editing a target nucleic acid sequence, or modulating expression of a target nucleic acid sequence.

### BACKGROUND

Adoptive cell therapies such as adoptive T cell therapy utilizing genetically modified T cells has entered clinical testing for solid and hematologic malignancies. In phase I and II trials involving hematologic malignancies (e.g. lymphoma, Chronic lymphocytic leukemia (CLL) and Acute lymphocytic leukemia (ALL)), many patients have exhibited at least a partial response, with some exhibiting complete responses (Kochenderfer, J. N. et al., 2012 Blood 119, 2709-2720). Improved methods and therapies are needed, including for use in solid tumor types, such as melanoma, renal cell carcinoma and colorectal cancer. See Johnson, L. A. et al., 2009 Blood 114, 535-546; Lamers, C. H. et al., 2013 Mol. Ther. 21, 904-912; Warren, R. S. et al., 1998 Cancer Gene Ther. 5, S1-S2). Among the provided embodiments are those addressing such need.

### SUMMARY

Provided herein are genome editing systems and related compositions and methods for the targeted editing of the nucleic acid sequence of CBLB. In certain embodiments, such targeted editing results in the alteration of CBLB expression. In certain embodiments, such alteration of expression occurs in T cells. In certain embodiments, the alteration of CBLB expression in T cells involves the use of a ribonucleoprotein (RNP) complex as a genome editing system comprising an RNA-guided nuclease protein complexed with a gRNA targeting the CBLB gene. In certain embodiments, the alteration in CBLB expression occurs as a result of a double-stranded break induced by the RNP and subsequent imperfect repair that leads to indels at and/or adjacent to the targeted CBLB sequence.

In certain embodiments, the instant disclosure relates to genome editing systems that include a guide RNA with a targeting domain that is complementary to target sequence of a CBLB gene and where the RNA-guided nuclease is a Cas9 nuclease. The targeting domain may be 70%, 80%, 85%, 90%, 95%, or 100% complementary.

In certain embodiments, the target sequence of the CBLB gene comprises the sequence of exon 2, exon 4, or exon 5.

In certain embodiments, the target sequence of the CBLB gene comprises the sequence selected from the group consisting of SEQ ID NOs: 88-92.

In certain embodiments, the targeting domain has a length of 16, 17, 18, 19, 20, 21, 22, 23, 24, 25, or 26 nucleotides.

In certain embodiments, the targeting domain has at least 18 contiguous nucleotides that are complementary to the CBLB gene.

In certain embodiments, the targeting domain comprises a nucleotide sequence that is identical to, or differs by no more than 3 nucleotides from, a nucleotide sequence selected from SEQ ID NOS: 1 to 14. In certain embodiments, the targeting domain is configured to form a double strand break or a single strand break within about 500 bp, about 450 bp, about 400 bp, about 350 bp, about 300 bp, about 250 bp, about 200 bp, about 150 bp, about 100 bp, about 50 bp, about 25 bp, or about 10 bp of the CBLB target position.

In certain embodiments disclosed herein, the genome editing system is capable of altering CBLB gene by knocking out the expression of the CBLB gene or knocking down the expression of the CBLB gene.

In certain embodiments, the genome editing systems disclosed herein incorporate a gRNA comprising a targeting domain configured to target a coding region or a non-coding region of the CBLB gene, wherein said non-coding region comprises a promoter region, an enhancer region, an intron, the 3′ UTR, the 5′ UTR, or a polyadenylation signal region of said CBLB gene; and the coding region comprises, e.g., an early coding region of said CBLB gene.

In certain embodiments, the genome editing systems disclosed herein incorporate a targeting domain comprising a nucleotide sequence that is identical to, or differs by no more than 3 nucleotides from, a nucleotide sequence selected from SEQ ID NOS: 1-14. In certain embodiments, the targeting domain comprises a nucleotide sequence that is identical to, or differs by no more than 3 nucleotides from, a nucleotide sequence selected from the group consisting of: (a) SEQ ID NO: 3; (b) SEQ ID NO: 4; (c) SEQ ID NO: 8; (d) SEQ ID NO: 12; and (e) SEQ ID NO: 14.

In certain embodiments, the present disclosure relates to a composition comprising a gRNA molecule comprising a targeting domain that is complementary with a target sequence of a CBLB gene. In certain embodiments, the composition comprises one, two, three, or four gRNA molecules. In certain embodiments, the composition further comprises an RNA-guided nuclease, e.g., a Cas9 molecule. In certain embodiments, the targeting domain incorporated into such compositions comprises a nucleotide sequence that is identical to, or differs by no more than 3 nucleotides from, a nucleotide sequence selected from SEQ ID NOS: 1-14. In certain embodiments, the targeting domain comprises a nucleotide sequence that is identical to, or differs by no more than 3 nucleotides from, a nucleotide sequence selected from the group consisting of: (a) SEQ ID NO: 3; (b) SEQ ID NO: 4; (c) SEQ ID NO: 8; (d) SEQ ID NO: 12; and (e) SEQ ID NO: 14.

In certain embodiments, the present disclosure relates to a vector encoding a gRNA molecule comprising a targeting domain that is complementary with a target sequence of a CBLB gene. In certain embodiments, the vector further encodes for an RNA-guided nuclease, e.g., a Cas9 molecule. In certain embodiments, the targeting domain of the gRNA encoded by the vector comprises a nucleotide sequence that is identical to, or differs by no more than 3 nucleotides from, a nucleotide sequence selected from SEQ ID NOS: 1-14. In certain embodiments, the targeting domain comprises a nucleotide sequence that is identical to, or differs by no more than 3 nucleotides from, a nucleotide sequence selected from the group consisting of: (a) SEQ ID NO: 3; (b) SEQ ID NO: 4; (c) SEQ ID NO: 8; (d) SEQ ID NO: 12; and (e) SEQ ID NO: 14. In certain embodiments the vector is a viral vector. In certain embodiments, the vector is an adeno-associated virus (AAV) vector or a lentivirus (LV) vector.

In certain embodiments, the present disclosure is directed to a method of altering a CBLB gene in a cell, comprising administering to said cell one of: (i) a genome editing system comprising a gRNA molecule comprising a targeting domain that is complementary with a target sequence of said CBLB gene, and a Cas9 molecule; (ii) a vector comprising a polynucleotide encoding a gRNA molecule comprising a targeting domain that is complementary with a target sequence of said CBLB gene, and a polynucleotide encoding a Cas9 molecule; or (iii) a composition comprising a gRNA molecule comprising a targeting domain that is complementary with a target sequence of said CBLB gene, and a Cas9 molecule.

In certain embodiments, the present disclosure is directed to a cell comprising a genome editing system as described herein, a gRNA composition of as described herein, or a vector as described herein. In certain embodiments, the cell expresses CBLB. In certain embodiments, the cell is a T cell.

In certain embodiments, the gRNA and the RNA-guided nuclease comprise a ribonucleoprotein (RNP) complex.

In certain embodiments, the methods comprise administering two or more RNP complexes comprising distinct gRNAs.

In certain embodiments, the RNP complex comprise enzymatically active Cas9 (eaCas9) nucleases.

In certain embodiments, the RNP complex comprise eaCas9 nucleases that form double strand breaks in a target nucleic acid or form single strand breaks in a target nucleic acid.

In certain embodiments, two RNP complexes comprising distinct gRNAs are used to form offset single strand breaks in the CBLB gene in the cell.

In certain embodiments, the present disclosure is directed to an RNA-guided nuclease-mediated method of altering CBLB gene expression in a cell comprising: a) contacting the cell with a sufficient amount of a gRNA that targets CBLB and an RNA-guided nuclease; and b) forming a first DNA double strand break at or near a CBLB target position in a CBLB gene of the cell, wherein the first DNA double strand break is repaired by NHEJ, wherein said repair alters the expression of the CBLB gene.

In certain embodiments, the method further comprises forming a second DNA double strand break at or near the CBLB target position.

In certain embodiments, the first double strand break is formed within about 500 bp, about 450 bp, about 400 bp, about 350 bp, about 300 bp, about 250 bp, about 200 bp, about 150 bp, about 100 bp, about 50 bp, about 25 bp, or about 10 bp of a CBLB target position.

In certain embodiments, the first and second double strand breaks are formed within about 500 bp, about 450 bp, about 400 bp, about 350 bp, about 300 bp, about 250 bp, about 200 bp, about 150 bp, about 100 bp, about 50 bp, about 25 bp, or about 10 bp of a CBLB target position.

In certain embodiments, the first double strand break is formed in a coding region or a non-coding region of said CBLB gene, wherein said non-coding region comprises a promoter region, an enhancer region, an intron, a 3′ UTR, a 5′ UTR, or a polyadenylation signal region of said CBLB gene.

In certain embodiments, the first and second double strand breaks are formed in a coding region or a non-coding region of said CBLB gene, wherein said non-coding region comprises a promoter region, an enhancer region, an intron, a 3′ UTR, a 5′ UTR, or a polyadenylation signal region of said CBLB gene.

In certain embodiments, said coding region is selected from exon 2, exon 4, and exon 5.

In certain embodiments, said targeting domain comprises a nucleotide sequence that is identical to, or differs by no more than about 3 nucleotides from, a nucleotide sequence selected from the group consisting of SEQ ID NOS: 1 to 14.

In certain embodiments, said RNA-guided nuclease is an S. pyogenes Cas9 nuclease, and said targeting domain comprises a nucleotide sequence that is identical to, or differs by no more than about 3 nucleotides from, a nucleotide sequence selected from the group consisting of: (a) SEQ ID NO: 3; (b) SEQ ID NO: 4; (c) SEQ ID NO: 8; (d) SEQ ID NO: 12; and (e) SEQ ID NO: 14.

In certain embodiments, said RNA-guided nuclease is an S. aureus Cas9 nuclease.

In certain embodiments, said RNA-guided nuclease is a mutant Cas9 nuclease.

In certain embodiments, the NHEJ repair produces an insertion or deletion with a frequency of greater than or equal to 20%.

In certain embodiments, the insertion or deletion frequency is greater than or equal to 30%, 40%, or 50%.

In certain embodiments, the present disclosure is directed to a genome engineered cell comprising an insertion or deletion near or at a target position of a CBLB gene, wherein said target position comprises a nucleotide sequence that is complementary to, or differs by no more than about 3 nucleotides from, a nucleotide sequence selected from SEQ ID NOS: 1 to 14.

In certain embodiments, the insertion or deletion is within about 500 bp, about 450 bp, about 400 bp, about 350 bp, about 300 bp, about 250 bp, about 200 bp, about 150 bp, about 100 bp, about 50 bp, about 25 bp, or about 10 bp of a CBLB target position.

In certain embodiments, the cell is a T cell or NK cell.

In certain embodiments, the cell further comprises an eTCR or a CAR.

In certain embodiments, the present disclosure is directed to a composition comprising: a) a population of cells comprising a CBLB gene comprising an insertion or deletion at or near a CBLB target position, wherein said CBLB target position comprises a nucleotide sequence that is complementary to, or differs by no more than about 3 nucleotides from, a nucleotide sequence selected from SEQ ID NOS: 1 to 14; and b) a storage buffer.

In certain embodiments, the population of cells comprises T cells or NK cells.

In certain embodiments, the T cells or NK cells further comprise an eTCR or a CAR.

In certain embodiments, the present disclosure is directed to a method of treating cancer in subject, comprising administering to the subject engineered immune cells, wherein the engineered immune cells have reduced expression of a CBLB gene, and optionally an engineered T Cell Receptor (eTCR) or a Chimeric Antigen Receptor (CAR), wherein the engineered immune cells have an insertion or a deletion near the CBLB gene.

In certain embodiments, the engineered immune cells comprise T cells or NK cells.

In certain embodiments, the eTCR or CAR has antigen specificity to a cancer cell.

In certain embodiments, CBLB expression in the engineered immune cells is reduced by introducing into the immune cells a genome editing system comprising a gRNA comprising a targeting domain that is complementary with a target sequence of said CBLB gene, and a RNA-guided nuclease.

In certain embodiments, the T cells are CD4+ T cells and/or CD8+ T cells.

In certain embodiments, the engineered immune cells maintain or have enhanced proliferation in the absence of CD28 co-stimulation relative to a non-engineered immune cell.

In certain embodiments, the engineered immune cells maintain or have enhanced proliferation in the absence of cytokines relative to non-engineered immune cells.

In certain embodiments, the engineered immune cells maintain or have increased expression of IFN-gamma, IL-2, and TNF-alpha relative to non-engineered immune cells.

In certain embodiments, the engineered immune cells maintain or have increased target cell killing capacity relative to non-engineered immune cells.

In certain embodiments, the present disclosure is directed to a method of enhancing the proliferation of immune cells in which CD28 co-stimulation is reduced or absent, comprising introducing into the immune cells a genome editing system comprising a gRNA molecule comprising a targeting domain that is complementary with a target sequence of said CBLB gene, and a RNA-guided nuclease, and reducing CBLB expression in the immune cells.

In certain embodiments, the method further comprises enhancing proliferation in the absence or reduction of cytokines.

In certain embodiments, there is an absence or reduction of the cytokines IL-2, IL-7, and IL-15.

In certain embodiments, the present disclosure is directed to a composition comprising a plurality of engineered T cells, wherein said engineered T cells exhibit reduced CBLB gene expression relative to non-engineered T cells.

In certain embodiments, the engineered T cells exhibit a CBLB gene expression level that is about 50%, about 40%, about 30%, about 20%, about 10% or about 5% the level of CBLB expression in non-engineered T cells.

In certain embodiments, the engineered T cells further comprise expression of an eTCR or a CAR.

In certain embodiments, the T cells are CD4+ T cells and/or CD8+ T cells.

In certain embodiments, the engineered T cells are further characterized by possessing one or more of: a) maintained or increased proliferation in the absence of CD28 co-stimulation; b) maintained or increased target cell killing in the absence of CD28 co-stimulation; c) greater sensitivity to a target antigen; d) maintained or increased target cell killing in the presence of reduced target antigen; and e) an increased ability to produce cytokines.

In certain embodiments, the present disclosure is directed to a composition comprising a plurality of engineered T cells, wherein said engineered T cells exhibit reduced CBLB gene expression relative to non-engineered T cells, said engineered T cells produced by contacting non-engineered T cells with a genome editing system comprising: a gRNA comprising a targeting domain that is complementary with a target sequence of a CBLB gene; and an RNA-guided nuclease.

In certain embodiments, the engineered T cells are further transduced with a vector that expresses an eTCR or a CAR.

In certain embodiments, the vector is a viral vector.

In certain embodiments, the viral vector is an adeno-associated virus (AAV) vector or a lentivirus (LV) vector.

In certain embodiments, said RNA-guided nuclease is an S. pyogenes Cas9 nuclease, and said targeting domain comprises a nucleotide sequence that is identical to, or differs by no more than about 3 nucleotides from, a nucleotide sequence selected from the group consisting of: (a) SEQ ID NO: 3; (b) SEQ ID NO: 4; (c) SEQ ID NO: 8; (d) SEQ ID NO: 12; and (e) SEQ ID NO: 14.

In certain embodiments, the present disclosure is directed to a genome editing system comprising: a first gRNA that targets a casitas B-lineage lymphoma proto-oncogene-b (CBLB) gene; a second gRNA that targets a T cell receptor alpha constant (TRAC) gene; a third gRNA that targets a T cell receptor beta constant (TRBC) gene; and an RNA-guided nuclease.

In certain embodiments, the present disclosure is directed to a composition comprising: a first gRNA that targets a CBLB gene; a second gRNA that targets a TRAC gene; and a third gRNA that targets a TRBC gene.

In certain embodiments, the present disclosure is directed to a method of treating cancer in a subject, comprising administering to the subject engineered immune cells, wherein the engineered immune cells have reduced CBLB gene expression, reduced TRAC gene expression, and reduced TRBC expression.

In certain embodiments, the engineered immune cells express an engineered T Cell Receptor (eTCR) or a Chimeric Antigen Receptor (CAR).

In certain embodiments, the eTCR or CAR has specificity to a cancer antigen.

In certain embodiments, the present disclosure is directed to an engineered immune cell comprising: a CBLB gene knockout; a TRAC gene knockout; and a TRBC gene knockout.

In certain embodiments, the present disclosure is directed to an engineered immune cell comprising: a CBLB gene knockdown; a TRAC gene knockdown; and a TRBC gene knockdown.

In certain embodiments, the present disclosure is directed to an engineered immune cell comprising: a CBLB gene knockout or knockdown; a TRAC gene knockout or knockdown; and a TRBC gene knockout or knockdown.

In certain embodiments, the present disclosure is directed to a method of producing an engineered immune cell having an insertion or deletion disrupting a CBLB gene, a TRAC gene, and a TRBC gene, comprising: i) isolating an immune cell; and ii) contacting the immune cell with a genome editing system comprising a first gRNA targeting a CBLB gene, a second gRNA targeting a TRAC gene, a third gRNA targeting a TRBC gene, and a RNA-guided nuclease to generate an engineered immune cell.

In certain embodiments, the cells further comprise an engineered T Cell Receptor (eTCR) or a Chimeric Antigen Receptor (CAR).

In certain embodiments, the present disclosure is directed to an engineered immune cell comprising (a) a recombinant receptor that specifically binds to an antigen and (b) a genetic disruption of a CBLB gene, said genetic disruption preventing or reducing the expression of a CBLB polypeptide, wherein: at least about 70%, at least about 75%, or at least about 80% or at least or greater than about 90% of the cells in the composition contain the genetic disruption; do not express the endogenous CBLB polypeptide; do not contain a contiguous CBLB gene, do not contain a CBLB gene, and/or do not contain a functional CBLB gene; and/or do not express a CBLB polypeptide; and/or at least about 70%, at least about 75%, or at least about 80% or at least or greater than about 90% of the cells in the composition that express the recombinant receptor contain the genetic disruption, do not express the endogenous CBLB polypeptide, and/or do not express a CBLB polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Headings, including numeric and alphabetical headings and subheadings, are for organization and presentation and are not intended to be limiting.

Other features and advantages of the invention will be apparent from the detailed description, drawings, and from the claims.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure. The patent or application file contains at least one drawing executed in color. Copies of this patent or patent application publication with color drawing(s) will be provided by the Office upon request and payment of the necessary fee.

FIG. 1 depicts primary screening data of exemplary S. pyogenes Cas9 gRNAs targeting Exons 2-5 of CBLB and their associated % indel or % frameshift frequency.

FIG. 2 depicts primary screening data of exemplary S. aureus Cas9 gRNAs targeting Exons 2-5 of CBLB and their associated % indel or % frameshift frequency.

FIG. 3 depicts a secondary, confirmation screen of exemplary S. pyogenes Cas9 gRNAs used at concentrations of 0.23, 0.72, and 2.27 μM for the Cas9/gRNA RNP and their associated average indel fraction.

FIG. 4 depicts two select gRNAs, used in the 2-part format or a single gRNA molecule. % indel frequency is shown at various RNP concentrations in nM.

FIG. 5 depicts % cutting of an in vitro CBLB template of several exemplary gRNAs at increasing RNP concentrations in μM.

FIG. 6A-FIG. 6C depict knockdown and editing efficiency with several exemplary gRNAs. Knockdown of CBLB protein was assessed by western blot (FIG. 6A) and the % reduction in CBLB expression was determined (FIG. 6B). NGS data is depicted showing % indel fractions and % frameshift fractions of the same gRNAs (FIG. 6C).

FIG. 7A-FIG. 7B depict intracellular staining of CBLB, analyzed by flow cytometry. A shift to the left of the graph indicates reduced intracellular staining of CBLB. Gene-editing was performed with several exemplary gRNAs in CD4+ (FIG. 7A) and CD8+ (FIG. 7B) T cells.

FIG. 8A-FIG. 8B depict Cell Trace Violet (CTV) FACS analysis of cell proliferation in a CBLB gene-edited background in CD4+ (FIG. 8A) and CD8+ (FIG. 8B) T cells. The cells were grown without anti-CD28 and without added cytokines, and with sub-optimal concentrations of plate bound anti-CD3 antibodies (1.0 μg/ml).

FIG. 9A-FIG. 9B depict proliferation of CD4+ (FIG. 9A) and CD8+ (FIG. 9B) T cells in a CBLB gene-edited background. The cells were grown in the presence of soluble anti-CD28 antibodies (1.0 μg/ml), with and without added cytokines, and in the presence of increasing concentrations of plate-bound anti-CD3 antibodies.

FIG. 10A-FIG. 10B depict proliferation of CD4+ (FIG. 10A) and CD8+ (FIG. 10B) T cells in a CBLB gene-edited background. The cells were grown without anti-CD28 antibodies, with and without added cytokines, and in the presence of increasing concentrations of plate-bound anti-CD3 antibodies.

FIG. 11A-FIG. 11C depict INFγ (FIG. 11A), IL-2 (FIG. 11B), and TNF-α (FIG. 11C) levels in CD8+ T cells in a CBLB gene-edited background. The cells were cultured without anti-CD28 co-stimulation, without added cytokines, and in the presence of decreasing concentrations of plate-bound anti-CD3 antibodies.

FIG. 12A-FIG. 12C depict INFγ (FIG. 12A), IL-2 (FIG. 12B), and TNF-α (FIG. 12C) levels in CD4+ T cells in a CBLB gene-edited background. The cells were cultured without co-stimulation, without added cytokines, and in the presence of decreasing concentrations of plate-bound anti-CD3 antibodies.

FIG. 13 depicts western blot analysis of CBLB protein levels in engineered (eTCR) transduced T cells with CBLB gene-edited backgrounds compared to unedited controls.

FIG. 14 depicts flow cytometry results for tetramer binding and a surrogate marker for HPV E7 specific TCR expression in T cells 11 days after transduction.

FIG. 15 depicts % caspase positive peptide pulsed T2 cells after incubation with CBLB gene-edited HPV E7 eTCR transduced T cells. Increasing HPV E7 peptide concentrations were used.

FIG. 16 depicts peptide pulsed T2 cell target killing by CBLB gene-edited HPV E7 eTCR transduced T cells. A concentration of 1000 nM, 10 nM, and 0.1 nM of HPV E7 peptide was used. Cells were incubated with or without CTLA4-Ig (2 μg/ml).

FIG. 17 depicts INFγ production by CBLB gene-edited HPV E7 eTCR transduced T cells. A concentration of 1000 nM, 10 nM, and 0.1 nM of HPV E7 peptide was used. Cells were incubated with or without CTLA4-Ig (2 μg/ml).

FIG. 18 depicts SCC152 cell target killing by CBLB gene-edited HPV E7 eTCR transduced T cells. A T cell:SCC152 ratio (Effector:Target cell ratio) of 5:1, 2.5:1, and 1.25:1 was used.

FIG. 19 depicts INFγ production by CBLB gene-edited HPV E7 eTCR transduced T cells in the presence of SCC152 cells. Various T cell:SCC152 ratios were used.

FIG. 20 depicts CBLB gene-edited HPV E7 eTCR transduced T cell proliferation analyzed by FACS at day 6 post incubation with HPV E7 antigen. The cells were incubated with and without CD86 co-stimulation.

### DETAILED DESCRIPTION

While not wishing to be bound by any particular theory, response rates and therapeutic outcomes in connection with adoptive T cell therapies, such as in solid tumors, may be influenced by a number of factors. Such factors can include: (1) T cell proliferation following adoptive transfer; (2) T cell survival, such as survival that may be impaired by tumor environment factors such as induction of T cell apoptosis by factors in the target cell, e.g., cancer cell, environment; and (3) attributes indicative of T cell function, such as function that may be impaired by various factors such as inhibition of cytotoxic T cell function by inhibitory factors secreted by host immune cells and/or target cells, e.g., cancer cells. One or more of such factors, in turn, may be influenced by the activity of Casitas B-lineage lymphoma proto-oncogene-b (CBLB), an E3 ubiquitin ligase.

CBLB generally is considered to be ubiquitously expressed in leukocytes where it may regulate multiple signaling pathways, including in T cells, NK cells, B cells, and myeloid cells. A primary function of CBLB generally is negatively regulating immune cell costimulatory signals through co-stimulatory receptors. TCR stimulation in the absence of co-stimulation may lead to upregulation of CBLB, which in turn may inhibit downstream signaling events (Ltz-Nicolandoni et al. Frontiers in oncology. Vol 5. Article 58. 2015). In mice, CBLB deletion has been reported to induce autoimmunity and enhanced rejection of spontaneous and implanted tumors (Stromnes et al. J. Clin Invest 120: 3722-3734. 2010). Based on these results, further work has demonstrated a role for CBLB in immune checkpoint regulation (Zhou et al. Nature. 506:52-57. 2014).

Exemplary strategies for targeting other immune checkpoint regulators, such as CTLA-4 and PD-1, rely on target-specific inhibitory antibodies. This strategy has been effective in the context of targets that are membrane bound, but less effective for intracellular targets, such as CBLB. Current strategies to target CBLB for therapeutic purposes have been limited methods that temporarily inhibit CBLB expression or activity. For example strategies exist to reduce CBLB expression via siRNA transfection to induce an RNA interference response (Sachet et al. J Immunother Cancer. 3 (Suppl. 2): P172. 2015). Another approach is to use a small molecule inhibitor of CBLB (Agarwal et al. AACR; Cancer Res 2016; 76(14 Suppl.): Abstract nr 2228). While these methods effectively suppress CBLB activity, their transient nature would make them ineffective in an adoptive T cell therapy context, where stable repression must be maintained throughout the therapeutic time course. There are currently no permanent methods described to inhibit CBLB function. Gene editing offers an attractive alternative as it creates a stable, long-term inhibition of CBLB and its downstream pathways.

CRISPRs (Clustered Regularly Interspaced Short Palindromic Repeats) evolved in bacteria and archea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of an RNA-guided nuclease to a target sequence in the viral genome. The RNA-guided nuclease, in turn, cleaves and thereby silences the viral target.

Recently, the CRISPR/Cas9 system has been adapted for genome editing in eukaryotic cells. The introduction of site-specific double strand breaks (DSBs) allows for target sequence alteration through endogenous DNA repair mechanisms, for example non-homologous end joining (NHEJ) or homology-directed repair (HDR). CRISPR/Cas9 represents a promising avenue for addressing CBLB-mediated inhibition of T-cells in the context of tumor therapy, but to date no viable approaches for addressing this issue in T-cells for use in tumor therapy have been identified

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

The phrase “consisting essentially of” means that the species recited are the predominant species, but that other species may be present in trace amounts or amounts that do not affect structure, function or behavior of the subject composition. For instance, a composition that consists essentially of a particular species will generally comprise 90%, 95%, 96%, or more of that species.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below. An indel may produce insertions or deletions creating in-frame or out-of-frame mutations in the target sequence.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single-or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai et al. (Nat. Biotechnol. 34(5): 483 (2016), incorporated by reference herein) or by other means well known in the art. Genome editing outcomes may also be assessed by in situ hybridization methods such as the FiberCombTM system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Subject” means a human or non-human animal A human subject can be any age (e.g., an infant, child, young adult, or adult), and may suffer from a disease, or may be in need of alteration of a gene. Alternatively, the subject may be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g. suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | Aor C |
| R | A or G |
| Y | C orT/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

### Overview

The genome editing systems described herein generally include one or more gRNAs comprising targeting domains that are complimentary to one or more CBLB target sequences, which target sequences, in turn, include or are adjacent to protospacer adjacent motif (PAM) sequences recognized by one or more RNA-guided nucleases with which the one or more gRNAs are associated (e.g., complexed). Accordingly, the genome editing systems of this disclosure are directed, in a site-specific manner, to the one or more CBLB target sequences, and operate to introduce an alteration within or proximate to those CBLB target sequences.

Alterations introduced into, or proximate to, the CBLB target sites by the genome editing systems of this disclosure will, most commonly, include DNA single-strand breaks (SSBs or “nicks”) and/or double strand breaks (DSBs). Nicks and DSBs, in turn, are repaired by cells in a manner that may result in in the introduction of small indels or larger insertions or deletions at one or more CBLB target sites, deletions of sequences between two CBLB target sites, and/or insertions of sequences (particularly exogenous sequences introduced into cells via donor template oligonucleotides) into CBLB sites, or between two CBLB target sites in a manner that replaces an endogenous cellular DNA sequence between those target sites. However, in some cases, the genome editing systems introduce one or more of a point mutation (e.g. via cysteine deamination), a change in DNA marking (e.g. DNA methylation, histone acetylation or deacetylation, or other chromatin modifications), and/or recruitment of trans-acting factors such as transcription factors. Alternatively, genome editing systems of this disclosure may associate, in a durable (e.g. over an interval of weeks, months or longer) or transient (over an interval of seconds, minutes, hours, or days) manner, with one or more CBLB target sequences, thereby preventing association of other factors (particularly RNA polymerases, but also DNA polymerases, transcription factors, and/or other cis- or trans-acting factors that influence gene expression) with the CBLB target sequences. These and other modes of action of genome editing systems and their components are described in detail below under the headings “RNA-guided nucleases” and “Modifications of RNA-guided nucleases.”

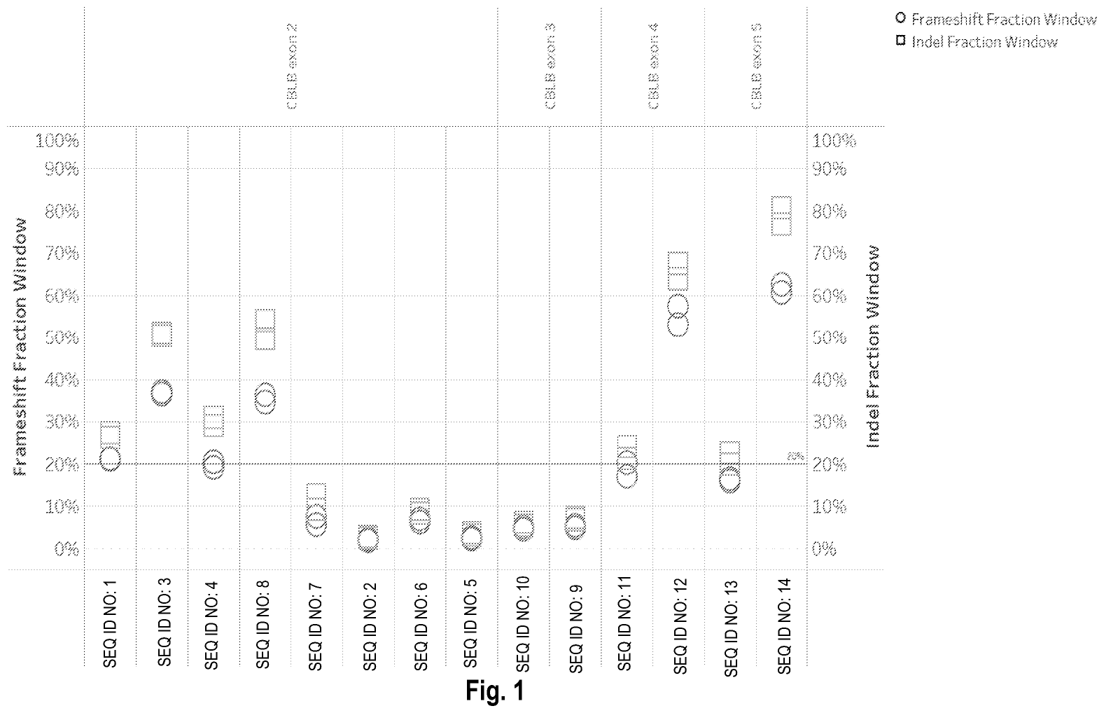

The CBLB target sequences and corresponding gRNA targeting domain sequences are generally, but not necessarily, located in exons, where the introduction of a small indel, or a larger insertion or deletion may result in one or more mutations (e.g., a frameshift mutation, a nonsense mutation, introduction of a codon for an amino acid that disrupts the structure of the surrounding protein, and/or removal of a codon for an amino acid that is necessary for protein activity) that reduce or eliminate function of the CBLB protein. FIG. 1 shows a mapping of cutting activity of various S. pyogenes guide RNAs to the locations they target within the exon structure of the CBLB gene. These mutations are referred to throughout this specification as “knockout” mutations, and their functional effect is “knockout” of CBLB protein function.

Certain CBLB target sequences may be considered “hot spot” target sites for gRNA targeting domain sequences. These are sites that are preferentially targeted because they produce high % indel frequencies or effective knock down or knock out of the CBLB gene. gRNAs targeting these preferred sites may produce % indel frequencies of 30% or higher. For example, a preferred target site in the CBLB gene may have complementary gRNA targeting domains that produce % indel frequencies of 30%, 40%, 50%, 60%, 70%, 80%, 85%, 90%, 95%, or higher. Hot spot target sites within a gene may not be readily apparent and require screening to identify them. Hot spot target sites within the TGFBR2 gene are described herein.

| CBLB Target Hot Spots | Sequence |
|---|---|
| SEQ ID NO: 88 | AAGCAAGCTGCCGCAGATCG |
| SEQ ID NO: 89 | TAAGCAAGCTGCCGCAGATC |
| SEQ ID NO: 90 | CTAAGCAAGCTGCCGCAGAT |
| SEQ ID NO: 91 | TGGAATTGACCATTGGGAAA |
| SEQ ID NO: 92 | TCATGAGGTCCACCAGATTA |

The CBLB target sequences can be, for example, located in exon 2, 4, or 5 of the CBLB gene. For example, but not by way of limitation, such exemplary targeting domains can comprises a nucleotide sequence that is identical to, or differs by no more than 1, 2, or 3 nucleotides from a nucleotide sequence selected from the group consisting of:
(a) SEQ ID NO: 3;
(b) SEQ ID NO: 4;
(c) SEQ ID NO: 8;
(d) SEQ ID NO: 12; and
(e) SEQ ID NO: 14.

As an alternative to knocking out CBLB expression, a transcriptional regulatory region, e.g., a promoter region (e.g., a promoter region that controls the transcription of the CBLB gene) can be targeted to alter (e.g., knock down) the expression of the gene. A targeted knockdown approach can be mediated by a CRISPR/Cas system comprising an enzymatically inactive Cas9 (eiCas9) molecule or an eiCas9 fusion protein (e.g., an eiCas9 fused to a transcription repressor domain or chromatin modifying protein), as described herein. For example, one or more gRNA molecules comprising a targeting domain can be configured to target an eiCas9 molecule or an eiCas9 fusion protein, sufficiently close to a transcriptional regulatory region, e.g., a promoter region (e.g., a promoter region that controls the transcription of CBLB gene), such that transcription of the CBLB gene is reduced and/or eliminated. .In certain embodiments, an eiCas9 or an eiCas9 fusion protein can be used to knock down CBLB expression in a T cell, e.g., a human T cell.

CBLB knock-out and/or knock down can be assessed in any suitable way, including without limitation the examination of the sequence of the CBLB gene, assessment of CBLB protein expression on the surface of cells (e.g. by immunostaining and cell sorting, particularly by fluorescence activated cell sorting or FACS including indirect intracellular staining flow cytometry), detection of cellular or molecular changes mediated by CBLB, or by western blot to detect CBLB protein levels. With respect to T cells in particular, CBLB knockout may be confirmed by (a) sequencing of the CBLB locus or T7E1 primer extension assay, and/or (b) intracellular FACS assessment of CBLB protein. Sequencing and T7E1 are described in greater detail below.

Knock out and/or knock down of CBLB may correspond to a 10%, 20%, 30%, 40%, 50%, 60%, 70%, 80%, 85%, 90%, 95%, or 100% reduction in CBLB expression relative to a baseline measurement or a wild-type cell.

In some aspects, the provided compositions and methods include those in which: at least or greater than about 50%, 60%, 65%, 70%, 75%, 80%, 85%, 90% or 95% of cells in a composition of cells into which an agent (e.g. gRNA/Cas9) for knockout or genetic disruption of a CBLB gene was introduced contain the genetic disruption; do not express the endogenous CBLB polypeptide; do not contain a contiguous CBLB gene, a CBLB gene, and/or a functional CBLB gene. In some embodiments, the methods, compositions and cells according to the present disclosure include those in which at least or greater than about 50%, 60%, 65%, 70%, 75%, 80%, 85%, 90% or 95% of cells in a composition of cells into which an agent (e.g. gRNA/Cas9) for knockout or genetic disruption of a CBLB gene was introduced do not express a CBLB polypeptide, such as on the surface of the cells. In some embodiments, at least or greater than about 50%, 60%, 65%, 70%, 75%, 80%, 85%, 90% or 95% of cells in a composition of cells into which an agent (e.g. gRNA/Cas9) for knockout or genetic disruption of a CBLB gene was introduced are knocked out in both alleles, i.e. comprise a biallelic deletion, in such percentage of cells.

Genome editing systems targeting CBLB may be implemented in a variety of ways, and their implementation may be tailored to the setting in which cells will be edited. Certain embodiments of this disclosure involve the delivery of RNA-guided nucleases and guide RNAs targeting CBLB to cells ex vivo in the form of ribonucleoprotein (RNP) complexes by means of electroporation, e.g., using electroporators and cuvettes available from commercial suppliers such as MaxCyte (Gaithersburg, MD) or Lonza (Basel, Switzerland). Other embodiments, however, may implement in vivo nucleic acids vectors, such as viral vectors or lipid nanoparticles, for either in vivo or ex vivo editing. Details of these implementations are described in greater detail below, under the heading “Implementation of genome editing systems.”

Knock-out and/or knock down of CBLB may be useful in a variety of settings, including without limitation in the context of adaptive T-cell therapy. According to certain embodiments of this disclosure, CBLB is knocked out in an immune cell, such as a T-cell, that will be used in therapy. As one example, the T-cell may express an engineered receptor such as a chimeric antigen receptor (CAR) or a heterologous T-cell receptor (TCR), which receptor may be configured to recognize an antigen on a cell or tissue that is implicated in a pathology such as a tumor cell. Whether or not they express an engineered receptor, CBLB knockout T-cells according the present disclosure may be employed in the targeting of a tissue or organ in which co-stimulatory signaling is limited or absent, T cell growth-promoting cytokines are limited or absent, and/or TCR signaling is sub-optimal.

CBLB knock-out and/or knock down cells may be employed in “autologous” cell therapies, in which cells are harvested from a subject, altered to knock-out or knock-down CBLB expression, and then returned to the same subject; alternatively, these cells may be administered to a different subject in an “allogeneic” cell therapy. In either approach, between harvesting and administration CBLB cells of this disclosure may be manipulated in a variety of ways, such as expanded, stimulated, purified or sorted, transduced with a transgene, frozen and/or thawed.

Knocking out or knocking down the CBLB gene as described herein can: (1) improve T cell proliferation; (2) improve T cell survival; and/or (3) improve T cell function. Knocking down the expression of the CBLB gene as described herein can similarly (1) improve T cell proliferation; (2) improve T cell survival; and/or (3) improve T cell function. Knocking out or knocking down the CBLB may improve these T cell parameters particularly where co-stimulation and cytokine levels are reduced or absent.

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation. In certain embodiments, the double strand or single strand break is within about 500 bp, about 450 bp, about 400 bp, about 350 bp, about 300 bp, about 250 bp, about 200 bp, about 150 bp, about 100 bp, about 50 bp, about 25 bp, or about 10 bp of a CBLB target position, thereby inducing an alteration in the expression of the CBLB gene.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i e flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant,” incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g. fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g. GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science Aug. 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, February 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains may contain one or more poly-A tracts, which can be recognized by RNA polymerases as a termination signal. The sequence of the first and second complentarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate that, although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

Table 2, below, provides exemplary gRNAs for targeting CBLB with an S. pyogenes Cas9.

TABLE 2-S
S. pyogenes gRNAs

| SEQ ID | SEQUENCE |
|---|---|
| SEQ ID | AGCAAGCTGCCGCAGATCGC |
| NO: 1 | |
| SEQ ID | TACCCAAAATTCGACCTTTT |
| NO: 2 | |
| SEQ ID | CGATCTGCGGCAGCTTGCTT |
| NO: 3 | |
| SEQ ID | GATCTGCGGCAGCTTGCTTA |
| NO: 4 | |
| SEQ ID | GGCAGAAACCCTGGTGGTCG |
| NO: 5 | |
| SEQ ID | GGATTTCCTCCTCGACCACC |
| NO: 6 | |
| SEQ ID | GGGTATTATTGATGCTATTC |
| NO: 7 | |
| SEQ ID | ATCTGCGGCAGCTTGCTTAG |
| NO: 8 | |
| SEQ ID | CGTAAATGCTGATATGTATC |
| NO: 9 | |
| SEQ ID | CCCGTTTTGACTTTTTCATA |
| NO: 10 | |
| SEQ ID | TGATACGAAAGTTATCTCCC |
| NO: 11 | |
| SEQ ID | TTTCCCAATGGTCAATTCCA |
| NO: 12 | |
| SEQ ID | CCACCAGATTAGCTCTGGCC |
| NO: 13 | |
| SEQ ID | TAATCTGGTGGACCTCATGA |
| NO: 14 | |

Table 3, below, provides exemplary gRNAs for targeting CBLB with an S. aureus Cas9.

TABLE 3
S. aureus gRNAs

| SEQ ID | SEQUENCE |
|---|---|
| SEQ ID | TGGCAGAAACCCTGGTGGTCG |
| NO: 15 | |
| SEQ ID | ATGGCAGAAACCCTGGTGGTC |
| NO: 16 | |
| SEQ ID | GGGGATTTCCTCCTCGACCAC |
| NO: 17 | |
| SEQ ID | AATCCCCGAAAAGGTCGAATT |
| NO: 18 | |
| SEQ ID | CGATCTGCGGCAGCTTGCTTA |
| NO: 19 | |
| SEQ ID | ATACCCAAAATTCGACCTTTT |
| NO: 20 | |
| SEQ ID | CTTAGGGGGTCCAACTGCATC |
| NO: 21 | |
| SEQ ID | TCGCAGGACCGTGGAGAAGAC |
| NO: 22 | |
| SEQ ID | GCAGAAACCCTGGTGGTCGAG |
| NO: 23 | |
| SEQ ID | TGGTGGTCGAGGAGGAAATCC |
| NO: 24 | |
| SEQ ID | GCTGCCGCAGATCGCAGGACC |
| NO: 25 | |
| SEQ ID | AGGAGGAAATCCCCGAAAAGG |
| NO: 26 | |
| SEQ ID | TGCGATCTGCGGCAGCTTGCT |
| NO: 27 | |
| SEQ ID | GCCGCAGATCGCAGGACCGTG |
| NO: 28 | |
| SEQ ID | GCCATTCATTGAGTTTGCCAT |
| NO: 29 | |
| SEQ ID | GTGGAGAAGACTTGGAAGCTC |
| NO: 30 | |
| SEQ ID | CAGAAACCCTGGTGGTCGAGG |
| NO: 31 | |
| SEQ ID | CTGCCGCAGATCGCAGGACCG |
| NO: 32 | |
| SEQ ID | CACCAGGGTTTCIGCCATTCA |
| NO: 33 | |
| SEQ ID | TACCCAAAATTCGACCTTTTC |
| NO: 34 | |
| SEQ ID | TTGATGCTATTCAGGATGCAG |
| NO: 35 | |
| SEQ ID | TAAGCAAGCTGCCGCAGATCG |
| NO: 36 | |
| SEQ ID | CGCAGGACCGTGGAGAAGACT |
| NO: 37 | |
| SEQ ID | TTGGGTATTATTGATGCTATT |
| NO: 38 | |
| SEQ ID | GCGATCTGCGGCAGCTTGCTT |
| NO: 39 | |
| SEQ ID | AAATTCCAGATGGCAAACTCA |
| NO: 40 | |
| SEQ ID | AATACCCAAAATTCGACCTTT |
| NO: 41 | |
| SEQ ID | AACTTGCCCAACTCAGTGAGA |
| NO: 42 | |
| SEQ ID | AAAGTACTCATTCTCACTGAG |
| NO: 43 | |
| SEQ ID | ATTCTCTCCTTGCCTTCTTTA |
| NO: 44 | |
| SEQ ID | AGAATGTATGAAGAACAGTCA |
| NO: 45 | |
| SEQ ID | ACTCIlIAAAGAAGGCAAGGA |
| NO: 46 | |
| SEQ ID | TAAGACICIlTAAAGAAGGCA |
| NO: 47 | |
| SEQ ID | GCAAAATATCAAGTATATATG |
| NO: 48 | |
| SEQ ID | AAAATCTACATTGATAGCCTT |
| NO: 49 | |
| SEQ ID | AACGGGCAATAAGACICIIIA |
| NO: 50 | |
| SEQ ID | TATCAGCATTTACGACTTATA |
| NO: 51 | |
| SEQ ID | ATATGGTGGGCTATTTTTCAA |
| NO: 52 | |
| SEQ ID | AGACICTTIAAAGAAGGCAAG |
| NO: 53 | |
| SEQ ID | TGCCAAAATCCCAAACTTCAG |
| NO: 54 | |
| SEQ ID | ATTTTAAAGTACTCATTCTCA |
| NO: 55 | |
| SEQ ID | TTGATTICIGCCAGCATGTGA |
| NO: 56 | |
| SEQ ID | GTGATACGAAAGTTATCTCCC |
| NO: 57 | |
| SEQ ID | TATCTCCCTGGAATTGACCAT |
| NO: 58 | |
| SEQ ID | CTGAAGATAAGGGACAGTTTT |
| NO: 59 | |
| SEQ ID | TGTGATACGAAAGTTATCTCC |
| NO: 60 | |
| SEQ ID | TTGTCTCCAAAAAACTTTCIC |
| NO: 61 | |
| SEQ ID | GTATCACAAAAGCAGATGCTG |
| NO: 62 | |
| SEQ ID | ATCTTTCCCAATGGTCAATTC |
| NO: 63 | |
| SEQ ID | TTATCTCCCTGGAATTGACCA |
| NO: 64 | |
| SEQ ID | CTTTCCCAATGGTCAATTCCA |
| NO: 65 | |
| SEQ ID | ATCTCCCTGGAATTGACCATT |
| NO: 66 | |
| SEQ ID | GTCCACCAGATTAGCTCTGGC |
| NO: 67 | |
| SEQ ID | TCCACCAGATTAGCTCTGGCC |
| NO: 68 | |
| SEQ ID | TGGACCTCATGAAGGCACTGT |
| NO: 69 | |
| SEQ ID | AGAGCTAATCTGGTGGACCTC |
| NO: 70 | |
| SEQ ID | TTAGAGCCATTGCTTCCAGGC |
| NO: 71 | |
| SEQ ID | AAGTATTCAGACAGTGCCTTC |
| NO: 72 | |

Guide RNA design and target-specific sequences for cancer immunotherapy purposes are described in more detail in WO2015161276, incorporated herein by reference.

gRNA Design

Methods for selection and validation of target sequences as well as off-target analyses have been described previously, e.g., in Mali; Hsu; Fu et al., 2014 Nat Biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

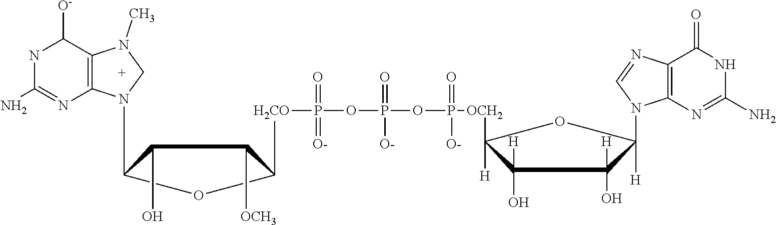

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

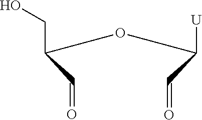

wherein “U” can be an unmodified or modified uridine.

The 3′ terminal U ribose can be modified with a 2′3′ cyclic phosphate as shown below:

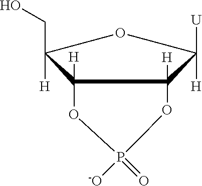

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino)

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with a-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains The REC lobe comprises an arginine-rich bridge helix (BH) domain, and at least one REC domain (e.g. a REC1 domain and, optionally, a REC2 domain) The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand. On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10× SYPRO Orange® (Life Technologies cat#S-6650) is dispensed into a 384 well plate. An equimolar amount of gRNA diluted in solutions with varied pH and salt is then added. After incubating at room temperature for 10′and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10× SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino.

Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e g flanking or adjoining) a target sequence to be cleaved. These homologous regions are referred to here as “homology arms,” and are illustrated schematically below:

[5′ homology arm]—[replacement sequence]—−[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication. In certain embodiments, the cell is a T cell. In certain embodiments, the cell is a CD4+ and/or a CD8+ T cell. In further embodiments, the cell is a T cell that expresses an engineered TCR (eTCR).

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 4 and 5 set forth several, non-limiting examples of genome editing system implementations. Those of skill in the art will appreciate, however, that these listings are not comprehensive, and that other implementations are possible. With reference to Table 4 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 4
Genome Editing System Components

| RNA-guided | | Donor | |
|---|---|---|---|
| Nuclease | Grna | Template | Comments |
| Protein | RNA | [N/A] | An RNA-guided nuclease protein |
| | | | complexed with a gRNA molecule (an |
| | | | RNP complex) |
| Protein | RNA | DNA | An RNP complex as described above |
| | | | plus a single-stranded or double |
| | | | stranded donor template. |
| Protein | DNA | [N/A] | An RNA-guided nuclease protein plus |
| | | | gRNA transcribed from DNA. |
| Protein | DNA | DNA | An RNA-guided nuclease protein plus |
| | | | gRNA-encoding DNA and a separate |
| | | | DNA donor template. |
| Protein | DNA | | An RNA-guided nuclease protein and |
| | | | a single DNA encoding both a gRNA |
| | | | and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a gRNA, |
| | | | and a second DNA or DNA vector |
| | | | encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| DNA | | | A first DNA or DNA vector encoding |
| | DNA | | an RNA-guided nuclease and a donor |
| | | | template, and a second DNA or DNA |
| | | | vector encoding a gRNA |
| DNA | | | A DNA or DNA vector encoding an |
| | RNA | | RNA-guided nuclease and a donor |
| | | | template, and a gRNA |

| | | |
|---|---|---|
| RNA | [N/A] | An RNA or RNA vector encoding an |
| | | RNA-guided nuclease and comprising |
| | | a gRNA |
| RNA | DNA | An RNA or RNA vector encoding an |
| | | RNA-guided nuclease and comprising |
| | | a gRNA, and a DNA or DNA vector |
| | | encoding a donor template. |

Table 5 summarizes various delivery methods for the components of genome editing systems, as described herein. Again, the listing is intended to be exemplary rather than limiting.

TABLE 5

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium Phosphate | | | | | and Proteins |
| transfection, cell compression or | | | | | |
| squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding elements of a genome editing system can include sequences that encode for one, two, three, four, or more gRNAs. For example the nucleic acids can encode for both a first and a second gRNA molecule, e.g., where the second gRNA has a second targeting domain that is complementary to a second target sequence of the CBLB gene. The nucleic acids disclosed herein can further comprise a nucleotide sequence that encodes a third gRNA molecule having a third targeting domain that is complementary to a third target sequence of the CBLB gene. The nucleic acid compositions disclosed herein can further comprise a nucleotide sequence that encodes a fourth gRNA molecule described herein having a fourth targeting domain that is complementary to a fourth target sequence of the CBLB gene.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 5, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 5, and additional suitable viral vectors and their use and production are described in Cotta-Ramusino. In certain embodiments, the vector is a viral vector, e.g., an adeno-associated virus (AAV) vector or a lentivirus (LV) vector. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) non-particles can be suitable for use as delivery vehicles in certain embodiments of this disclosure. Exemplary lipids for use in nanoparticle formulations, and/or gene transfer are shown in Table 6, and Table 7 lists exemplary polymers for use in gene transfer and/or nanoparticle formulations.

TABLE 6
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethy lammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CT AB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3-[N-(N’,N’-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethy lammonium propane | DMTAP | Cationic |
| 0,0 ‘-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CD AN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

TABLE 7
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3’-dithiobispropionimidate | DTBP |
| Poly(ethylene imine) biscarbamate | PEIC |
| Poly(L-lysine) | PLL |
| Histidine modified PLL | |
| Poly (N-vinylpyrrolidone) | PVP |
| Poly (propylenimine) | PPI |
| Poly (amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxy propylmethacrylamide) | pHPMA |
| Poly(2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases, i.e., ribonucleoprotein complexes) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

In certain embodiments, the RNP complexes of the present disclosure, including, e.g., RNP pharmaceutical compositions, can be used to: (1) improve T cell proliferation; (2) improve T cell survival; and/or (3) improve T cell function. For example, but not by way of limitation, two or more RNP complexes comprising distinct gRNAs can be employed concurrently or sequentially to alter CBLB gene expression in a cell, e.g., a T cell. Such RNP complexes can comprise distinct gRNAs targeting distinct CBLB gene sequences. The RNP complexes can, in certain instances, induce a cleavage event, e.g., a double strand or single strand break. For example, the RNP complexes can comprise enzymatically active Cas9 (eaCas9) molecules that form double strand breaks in a target nucleic acid or eaCas9 molecules that form single strand breaks in a target nucleic acid (e.g., nickase molecules). In certain embodiments, a dual-nickase RNP strategy can be used to form two offset single strand breaks that, in turn, form a single double strand break having an overhang (e.g. a 5′ overhang).

Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

Multi-Modal or Differential Delivery of Components

Skilled artisans will appreciate, in view of the instant disclosure, that different components of genome editing systems disclosed herein can be delivered together or separately and simultaneously or non-simultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e g , minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### Genetically Engineered Cells and Methods of Producing Cells Expressing a Recombinant Receptor

Provided herein are cells for adoptive cell therapy, e.g., adoptive immunotherapy, and methods for producing or generating the cells. The cells include immune cells such as T cells. The cells generally are engineered by introducing one or more genetically engineered nucleic acids or products thereof. Among such products are genetically engineered antigen receptors, including engineered T cell receptors (TCRs) and functional non-TCR antigen receptors, such as chimeric antigen receptors (CARs), including activating, stimulatory, and costimulatory CARs, and combinations thereof. In some embodiments, the cells also are introduced, either simultaneously or sequentially, with the nucleic acid encoding the genetically engineered antigen receptor, with an agent (e.g., Cas9/gRNA RNP) that is capable of disrupting a gene encoding CBLB.

In some embodiments, the cells (e.g., T cells) can be incubated or cultivated prior to, during and/or subsequent to, introducing the nucleic acid molecule encoding the recombinant receptor and/or the agent (e.g., Cas9/gRNA RNP). In some embodiments, the cells (e.g., T cells) can be incubated or cultivated prior to, during or subsequent to, the introduction of the nucleic acid molecule encoding the recombinant receptor, such as prior to, during or subsequent to, the transduction of the cells with a viral vector (e.g., a lentiviral vector) encoding the recombinant receptor. In some embodiments, the cells (e.g., T cells) can be incubated or cultivated prior to, during or subsequent to, the introduction of the agent (e.g., Cas9/gRNA RNP), such as prior to, during or subsequent to, contacting the cells with the agent or prior to, during or subsequent to, delivering the agent into the cells, e.g., via electroporation. In some embodiments, the incubation can be both in the context of introducing the nucleic acid molecule encoding the recombinant receptor and introducing the agent, e.g., Cas9/gRNA RNP. In some embodiments, the incubation can be in the presence of a cytokine, such as IL-2, IL-7 or IL-15, or in the presence of a stimulating or activating agent that are capable of inducing proliferation and/or activation of cells, such as an anti-CD³/anti-CD28 antibodies.

In some embodiments, the method includes activating or stimulating cells with a stimulating or activating agent (e.g., anti-CD³/anti-CD28 antibodies) prior to introducing the nucleic acid molecule encoding the recombinant receptor and the agent, e.g., Cas9/gRNA RNP. In some embodiments, incubation also can be performed in the presence of a cytokine, such as IL-2 (e.g., 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g., 0.1 ng/mL to 50 ng/mL, such as 0.5 ng/mL to 25 ng/mL, for example, at least or about 1 ng/mL or 5 ng/mL). In some embodiments, the cells are incubated for 6 hours to 96 hours, such as 24-48 hours or 24-36 hours prior to introducing the nucleic acid molecule encoding the recombinant receptor (e.g., via transduction).

Cells and Preparation of Cells for Genetic Engineering

Recombinant receptors that bind to a specific antigen and agents (e.g., Cas9/gRNA RNP) for gene editing of a CBLB gene encoding a CBLB polypeptide can be introduced into a wide variety of cells. In some embodiments, a recombinant receptor is engineered and/or the CBLB target gene is manipulated ex vivo and the resulting genetically engineered cells are administered to a subject. Sources of target cells for ex vivo manipulation may include, e.g., the subject's blood, the subject's cord blood, or the subject's bone marrow. Sources of target cells for ex vivo manipulation may also include, e.g., heterologous donor blood, cord blood, or bone marrow.

In some embodiments, the cells, e.g., engineered cells, are eukaryotic cells, such as mammalian cells, e.g., human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In some embodiments, the target cell is a T cell, e.g., a CD8+ T cell (e.g., a CD8+ naïve T cell, central memory T cell, or effector memory T cell), a CD4+ T cell (e.g., a CD4+ naïve T cell, central memory T cell, or effector memory T cell), a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naïve T cell, central memory T cell, or effector memory T cell), a CD4+ T cell (e.g., a CD4+ naïve T cell, central memory T cell, or effector memory T cell), a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the subtypes and subpopulations of T cells and/or of CD4+ and/or of CD8+ T cells, are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the methods include isolating cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from a mouse, a rat, a non-human primate, or a pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more properties, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects, contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++-free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker−) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Rα (CD127). In some examples, CD8+ T cells are enriched for cells positive for CD45RO (or negative for CD45RA) and for CD62L.

For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a peripheral blood mononuclear cell (PBMC) sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naïve, memory, and/or effector T cell subpopulations.

In some embodiments, CD8+ cells are further enriched for or depleted of naïve, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. (See Terakura et al. (2012) Blood.1:72-82; Wang et al. (2012) J Immunother. 35(9):689-701.) In some embodiments, combining TCM-enriched CD8+ T cells and CD4+ T cells further enhances efficacy or response.

In embodiments, memory T cells are present in both CD62L+and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naïve CD4+ T lymphocytes are CD45RO−, CD45RA+, CD62L+, CD4+ T cells. In some embodiments, central memory CD4+ cells are CD62L+and CD45RO+. In some embodiments, effector CD4+ cells are CD62L- and CD45RO.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate or stimulate the cells.

In some embodiments, the stimulating conditions or agents include one or more agent, e.g., ligand, which is capable of inducing signaling via an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the T cells are expanded by adding to the culture-initiating composition feeder cells, such as non-dividing PBMCs (e.g., such that the resulting population of cells contains at least about 5, 10, 20, or 40 or more PBMC feeder cells for each T lymphocyte in the initial population to be expanded), and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCLs) as feeder cells. LCLs can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the methods include re-introducing the engineered cells into the same patient, before or after cryopreservation.

### Recombinant Receptors

In some embodiments, the cells comprise one or more nucleic acids encoding a recombinant receptor introduced via genetic engineering, and genetically engineered products of such nucleic acids. In some embodiments, the cells can be produced or generated by introducing into a cell (e.g., via transduction of a viral vector, such as a retroviral or lentiviral vector) a nucleic acid molecule encoding the recombinant receptor. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, the target cell has been altered to bind to one or more target antigen, such as one or more tumor antigen. In some embodiments, the target antigen is selected from ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag. In some embodiments, the target cell has been altered to bind one or more of the following tumor antigens, e.g., by a TCR or a CAR. Tumor antigens may include, but are not limited to, AD034, AKT1, BRAP, CAGE, CDX2, CLP, CT-7, CT8/HOM-TES-85, cTAGE-1, Fibulin-1, HAGE, HCA587/MAGE-C2, hCAP-G, HCE661, HER2/neu, HLA-Cw, HOM-HD-21/Galectin9, HOM-MEEL-40/SSX2, HOM-RCC-3.1.3/CAXII, HOXA7, HOXB6, Hu, HUB1, KM-HN-3, KM-KN-1, KOC1, KOC2, KOC3, KOC3, LAGE-1, MAGE-1, MAGE-4a, MPP11, MSLN, NNP-1, NY-BR-1, NY-BR-62, NY-BR-85, NY-CO-37, NY-CO-38, NY-ESO-1, NY-ESO-5, NY-LU-12, NY-REN-10, NY-REN-19/LKB/STK11, NY-REN-21, NY-REN-26/BCR, NY-REN-3/NY-CO-38, NY-REN-33/SNC6, NY-REN-43, NY-REN-65, NY-REN-9, NY-SAR-35, OGFr, PLU-1, Rab38, RBPJkappa, RHAMM, SCP1, SCP-1, SSX3, SSX4, SSX5, TOP2A, TOP2B, or Tyrosinase.

### Antigen Receptors:

Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416,and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens that may be targeted by the receptors include, but are not limited to, αvβ6 integrin (avb6 integrin), B cell maturation antigen (BCMA), B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), Her2/neu (receptor tyrosine kinase erbB2), Her3 (erb-B3), Her4 (erb-B4), erbB dimers, human high molecular weight-melanoma-associated antigen (HMW-MAA), hepatitis B surface antigen, Human leukocyte antigen A1 (HLA-AI), human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1CAM), CE7 epitope of L1-CAM, Leucine Rich Repeat Containing 8 Family Member A (LRRC8A), Lewis Y, melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, preferentially expressed antigen of melanoma (PRAME), progesterone receptor, a prostate specific antigen, prostate stem cell antigen (PSCA), prostate specific membrane antigen (PSMA), receptor tyrosine kinase like orphan receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms tumor 1 (WT-1), and a pathogen-specific antigen.

In some embodiments, antigens targeted by the receptors in some embodiments include orphan tyrosine kinase receptor ROR1, tEGFR, Her2, Ll-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acetylcholine e receptor, GD2, GD3, HMW-MAA, IL-22R-alpha, IL-13R-alpha2, kdr, kappa light chain, Lewis Y, Ll-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

In some embodiments, the CAR has binding specificity for a tumor associated antigen, e.g., CD19, CD20, carbonic anhydrase IX (CAIX), CD171, CEA, ERBB2, GD2, alpha-folate receptor, Lewis Y antigen, prostate specific membrane antigen (PSMA) or tumor associated glycoprotein 72 (TAG72).

In some embodiments, the CAR binds a pathogen-specific antigen. In some embodiments, the CAR is specific for viral antigens (such as HIV, HCV, HBV, etc.), bacterial antigens, and/or parasitic antigens.

Among the chimeric receptors are chimeric antigen receptors (CARs). The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain.

Exemplary spacers include, but are not limited to, those described in Hudecek et al. (2013) Clin. Cancer Res., 19:3153 or international patent application publication number WO2014031687. In some embodiments, the spacer has the sequence set forth in SEQ ID NO: 74, and is encoded by the sequence set forth in SEQ ID NO: 73. In some embodiments, the spacer has the sequence set forth in SEQ ID NO: 75. In some embodiments, the spacer has the sequence set forth in SEQ ID NO: 76. In some embodiments, the constant region or portion is of IgD. In some embodiments, the spacer has the sequence set forth in SEQ ID NO:77. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 74, 75, 76 or 77.

TABLE 8
Exemplary spacer sequences

| SEQ ID NO: | Sequence |
|---|---|
| 73 | GAATCTAAGT ACGGACCGCC |
| | CTGCCCCCCT TGCCCT |
| 74 | ESKYGPPCPPCP |
| 75 | ESKYGPPCPPCPGQPREPQV |
| | YTLPPSQEEMTKNQVSLTCL |
| | VKGFYPSDIAVEWESNGQPE |
| | NNYKTTPPVLDSDGSFFLYS |
| | RLTVDKSRWQEGNVFSCSVM |
| | HEALHNHYTQKSLSLSLGK |
| 76 | ESKYGPPCPPCPAPEFLGG |
| | PSVFLFPPKPKDTLMISRTP |
| | EVTCVVVDVSQEDPEVQFNW |
| | YVDGVEVHNAKTKPREEQFN |
| | STYRVVSVLTVLHQDWLNGK |
| | EYKCKVSNKGLPSSIEKTIS |
| | KAKGQPREPQVYTLPPSQEE |
| | MTKNQVSLTCLVKGFYPSDI |
| | AVEWESNGQPENNYKTTPPV |
| | LDSDGSFFLYSRLTVDKSRW |
| | QEGNVFSCSVMHEALHNHYT |
| | QKSLSLSLGK |
| 77 | RWPESPKAQASSVPTAQPQA |
| | EGSLAKATTAPATTRNTGRG |
| | GEEKKKEKEKEEQEERETKT |
| | PECPSHTQPLGVYLLTPAVQ |
| | DLWLRDKATFTCFVVGSDLK |
| | DAHLTWEVAGKVPTGGVEEG |
| | LLERHSNGSQSQHSRLTLPR |
| | SLWNAGTSVTCTLNHPSLPP |
| | QRLMALREPAAQAPVKLSLN |
| | LLASSDPPEAASWLLCEVSG |
| | FSPPNILLMWLEDQREVNTS |
| | GFAPARPPPQPGSTTFWAWS |
| | VLRVPAPPSPQPATYTCVVS |
| | HEDSRTLLNASRSLEVSYVT |
| | DH |

This antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CDS, CD8, CD9, CD 16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s)

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ)or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, prolonged activation and full-blown immune response generally involve not only receiving a signal through the TCR complex, but also a costimulatory signal. Thus, in some embodiments, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation and responses are in some aspects described as being mediated by two classes of cytoplasmic signaling sequences: those that initiate signals characteristic of those following antigen-dependent primary signaling through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from the CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published Application No. WO 2014/031687. For example, the marker can be a truncated EGFR (tEGFR) that is optionally linked to a linker sequence, such as a T2A cleavable linker sequence. An exemplary polypeptide for a truncated EGFR (e.g. tEGFR) comprises the sequence of amino acids set forth in SEQ ID NO: 51218 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 79. An exemplary T2A linker sequence comprises the sequence of amino acids set forth in SEQ ID NO: 78 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 78.

TABLE 9
Truncated EGFR and T2A sequences

| SEQ ID NO | Sequence |
|---|---|
| 78 | LEGGGEGRGSLLTCGDVEENPGPR |
| 79 | MLLLVTSLLLCELPHPAFLLI |
| | PRKVCNGIGIGEFKDSLSIN |
| | ATNIKHFKNCTSISGDLHIL |
| | PVAFRGDSFTHTPPLDPQEL |
| | DILKTVKEITGFLLIQAWPE |
| | NRTDLHAFENLEIIRGRTKQ |
| | HGQFSLAVVSLNITSLGLRS |
| | LKEISDGDVIISGNKNLCYA |
| | NTINWKKLFGTSGQKTKIIS |
| | NRGENSCKATGQVCHALCSP |
| | EGCWGPEPRDCVSCRNVSRG |
| | RECVDKCNLLEGEPREFVEN |
| | SECIQCHPECLPQAMNITCT |
| | GRGPDNCIQCAHYIDGPHCV |
| | KTCPAGVMGENNTLVWKYAD |
| | AGHVCHLCHPNCTYGCTGPG |
| | LEGCPTNGPKIPSIATGMVG |
| | ALLLLLVVALGIGLFM |

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain In some embodiments, the chimeric antigen receptor includes a transmembrane domain linking the extracellular domain and the intracellular signaling domain. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 80 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:80; in some embodiments, the transmembrane-domain containing portion of the recombinant receptor comprises the sequence of amino acids set forth in SEQ ID NO: 81 or a sequence of amino acids having at least at or about 85%, 86%, 87%, 88%, 89%, 90%, 91%, 92%, 93%, 94%, 95%, 96%, 97%, 98%, 99% or more sequence identity thereto.

TABLE 10
Transmembrane domain sequences

| SEQ ID NO | Sequence |
|---|---|
| 80 | FWVLVVVGGVLACYSLLVTVAFIIFWV |
| 81 | IEVMYPPPYLDNEKSNGTIIHVKGKHL |
| | CPSPLFPGPSKPFWVLVVVGGVLACYS |
| | LLVTVAFIIFWV |

In some embodiments, the chimeric antigen receptor contains an intracellular domain of a T cell costimulatory molecule. In some aspects, the T cell costimulatory molecule is CD28 or 41BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 82 or 83 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 82 or 83. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 84 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 84.

TABLE 11
Intracellular signaling
domain sequences.

| SEQ | |
|---|---|
| ID | |
| NO | Sequence |
| 82 | RSKRSRLLHSDYMNMTPRRP |
| | GPTRKHYQPYAPPRDFAAYR |
| | S |
| 83 | RSKRSRGGHSDYMNMTPRRP |
| | GPTRKHYQPYAPPRDFAAYR |
| | S |
| 84 | KRGRKKLLYIFKQPFMRPVQ |
| | TTQEEDGCSCRFPEEEEGGC |
| | EL |

In some embodiments, the intracellular signaling domain comprises a human CD3 zeta stimulatory signaling domain or functional variant thereof, such as a 112 AA cytoplasmic domain of isoform 3 of human CD3ζ (Accession No.: P20963.2) or a CD3 zeta signaling domain as described in U.S. Pat. No.: 7,446,190 or U.S. Pat. No. 8,911,993. In some embodiments, the intracellular signaling domain comprises the sequence of amino acids set forth in SEQ ID NO: 85, 86 or 87 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 85, 86 or 87.

TABLE 12
Intracellular signaling domain sequences.

| SEQ ID NO | Sequence |
|---|---|
| 85 | RVKFSRSADAPAYQQGQNQL |
| | YNELNLGRREEYDVLDKRRG |
| | RDPEMGGKPRRKNPQEGLYN |
| | ELQKDKMAEAYSEIGMKGER |
| | RRGKGHDGLYQGLSTATKDT |
| | YDALHMQALPPR |
| 86 | RVKFSRSAEPPAYQQGQNQL |
| | YNELNLGRREEYDVLDKRRG |
| | RDPEMGGKPRRKNPQEGLYN |
| | ELQKDKMAEAYSEIGMKGER |
| | RRGKGHDGLYQGLSTATKDT |
| | YDALHMQALPPR |
| 87 | RVKFSRSADAPAYKQGQNQL |
| | YNELNLGRREEYDVLDKRRG |
| | RDPEMGGKPRRKNPQEGLYN |
| | ELQKDKMAEAYSEIGMKGER |
| | RRGKGHDGLYQGLSTATKDT |
| | YDALHMQALPPR |

In some aspects, the spacer contains only a hinge region of an IgG, such as only a hinge of IgG4 or IgG1, such as the hinge only spacer set forth in SEQ ID NO: 74. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only, such as set forth in SEQ ID NO: 75. In some embodiments, the spacer is or comprises a glycine-serine rich sequence or other flexible linker such as known flexible linkers.

For example, in some embodiments, the CAR includes an antibody or fragment that specifically binds an antigen, a spacer such as any of the Ig-hinge containing spacers, a CD28 transmembrane domain, a CD28 intracellular signaling domain, and a CD3 zeta signaling domain. In some embodiments, the CAR includes an antibody or fragment that specifically binds an antigen, a spacer such as any of the Ig-hinge containing spacers, a CD28 transmembrane domain, a CD28 intracellular signaling domain, and a CD3 zeta signaling domain. In some embodiments, such CAR constructs further includes a T2A ribosomal skip element and/or a tEGFR sequence, e.g., downstream of the CAR.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

### T Cell Receptors

In some embodiments, the genetically engineered antigen receptors include recombinant T cell receptors (TCRs) and/or TCRs cloned from naturally occurring T cells. Thus, in some embodiments, the target cell has been altered to contain specific T cell receptor (TCR) genes (e.g., a TRAC and TRBC gene). TCRs or antigen-binding portions thereof include those that recognize a peptide epitope or T cell epitope of a target polypeptide, such as an antigen of a tumor, viral or autoimmune protein. In some embodiments, the TCR has binding specificity for a tumor associated antigen, e.g., carcinoembryonic antigen (CEA), GP100, melanoma antigen recognized by T cells 1 (MART1), melanoma antigen A3 (MAGEA3), NYESO1 or p53.

In some embodiments, a “T cell receptor” or “TCR” is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. Generally, a TCR is or can be expressed on the surface of T cells (or T lymphocytes) where it is generally responsible for recognizing antigens bound to major histocompatibility complex (MHC) molecules.

In some embodiments, the TCR is a full-length TCR or antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex (see, e.g., Draper et al. Clin Cancer Res. 2015 Oct. 1; 21(19): 4431-4439, and US 20170145070 A1.)

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in super-antigen binding and not antigen recognition (Kotb (1995) Clinical Microbiology Reviews, 8:411-426).

In some embodiments, a TCR contains a variable alpha domain (V_{α}) and/or a variable beta domain (V_{β}) or antigen-binding fragments thereof. In some embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). In some embodiments, the α chain constant domain is encoded by the TRAC gene (IMGT nomenclature) or is a variant thereof. In some embodiments, the β chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Vα chains and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive.

In some embodiments, the TCR may be a heterodimer of two chains α and β (or optionally γ and δ) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the T-cells can be a cultured T-cell hybridoma or clone. In some embodiments, the TCR or antigen-binding portion thereof can be synthetically generated from knowledge of the sequence of the TCR.

In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808). In some embodiments, phage display is used to isolate TCRs against a target antigen (see, e.g., Varela-Rohena et al. (2008) Nat Med. 14:1390-1395 and Li (2005) Nat Biotechnol. 23:349-354).

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the α chain and β chain) that form a native inter-chain disulfide bond are substituted to another residue, such as to a serine or alanine. In some embodiments, an introduced disulfide bond can be formed by mutating non-cysteine residues on the alpha and beta chains, such as in the constant domain of the α chain and β chain, to cysteine. Exemplary non-native disulfide bonds of a TCR are described in published International PCT Nos. WO 2006/000830 and WO 2006/037960. In some embodiments, cysteines can be introduced at residue Thr48 of the α chain and Ser57 of the β chain, at residue Thr45 of the α chain and Ser77 of the β chain, at residue Tyr10 of the α chain and Ser17 of the β chain, at residue Thr45 of the α chain and Asp59 of the β chain and/or at residue Ser15 of the α chain and Glu15 of the β chain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR chains contain a transmembrane domain. In some embodiments, the transmembrane domain is positively charged. In some cases, the TCR chain contains a cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3δ chains) contain one or more immunoreceptor tyrosine-based activation motif or ITAM that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, for purposes of the provided methods, the TCR is in cell-bound form expressed on the surface of a cell.

In some embodiments, a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the inter-chain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable a domain, a constant a domain and a first dimerization motif attached to the C-terminus of the constant a domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR, which is a single amino acid strand containing an a chain and a β chain that is able to bind to MHC-peptide complexes. Typically, a scTCR can be generated using methods known to those of skill in the art, See e.g., International published PCT Nos. WO 1996/13593, WO 1996/18105, WO 1999/18129, WO 2004/033685, WO 2006/037960, WO 2011/044186; U.S. Pat. No. 7,569,664; and Schlueter, C. J. et al. J. Mol. Biol. 256, 859 (1996).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an amino acid sequence corresponding to a TCR β chain variable region, a second segment constituted by an amino acid sequence corresponding to a TCR α chain variable region sequence fused to the N terminus of an amino acid sequence corresponding to a TCR α chain constant domain extracellular sequence, and a linker sequence linking the C terminus of the first segment to the N terminus of the second segment.

In some embodiments, a scTCR contains a first segment constituted by an a chain variable region sequence fused to the N terminus of an a chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an a chain variable region sequence fused to the N terminus of a sequence α chain extracellular constant and transmembrane sequence, and, optionally, a linker sequence linking the C terminus of the first segment to the N terminus of the second segment.

In some embodiments, for the scTCR to bind an MHC-peptide complex, the α and β chains must be paired so that the variable region sequences thereof are orientated for such binding. Various methods of promoting pairing of an α and β in a scTCR are well known in the art. In some embodiments, a linker sequence is included that links the α and β chains to form the single polypeptide strand. In some embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-MHC complex.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P-, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduce bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- or -PGGG-(SGGGG)₆-P-, wherein P is proline, G is glycine and S is serine. In some embodiments, the linker has the sequence GSADDAKKDAAKKDGKS).

In some embodiments, a scTCR contains a disulfide bond between residues of the single amino acid strand, which, in some cases, can promote stability of the pairing between the α and β regions of the single chain molecule (see e.g. U.S. Pat. No. 7,569,664). In some embodiments, the scTCR contains a covalent disulfide bond linking a residue of the immunoglobulin region of the constant domain of the α chain to a residue of the immunoglobulin region of the constant domain of the β chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described above. In some cases, both a native and a non-native disulfide bond may be present.

In some embodiments, a scTCR is a non-disulfide linked truncated TCR in which heterologous leucine zippers fused to the C-termini thereof facilitate chain association (see e.g. International published PCT No. WO 1999/60120). In some embodiments, a scTCR contain a TCRα variable domain covalently linked to a TCRβ variable domain via a peptide linker (see e.g., International published PCT No. WO 1999/18129).

In some embodiments, any of the TCRs, including a dTCR or scTCR, can be linked to signaling domains that yield a functional TCR on the surface of a T cell. In some embodiments, the TCR is expressed on the surface of cells. In some embodiments, the TCR does contain a sequence corresponding to a transmembrane sequence. In some embodiments, the transmembrane domain can be a Cα or Cβ transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. In some embodiments, to generate a vector encoding a TCR, the α and β chains can be PCR amplified from total cDNA isolated from a T cell clone expressing the TCR of interest and cloned into an expression vector. In some embodiments, the α and β chains can be synthetically generated.

In some embodiments, the TCR alpha and beta chains are isolated and cloned into a gene expression vector. In some embodiments, transcription units can be engineered as a bicistronic unit containing an IRES (internal ribosome entry site), which allows co-expression of gene products (e.g. encoding an α and β chains) by a message from a single promoter. Alternatively, in some cases, a single promoter may direct expression of an RNA that contains, in a single open reading frame (ORF), multiple genes (e.g., encoding an α and β chains) separated from one another by sequences encoding a self-cleavage peptide (e.g., T2A) or a protease recognition site (e.g., furin). The ORF thus encodes a single polyprotein, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream. Examples of 2A cleavage peptides, including those that can induce ribosome skipping, are T2A, P2A, E2A and F2A. In some embodiments, the α and β chains are cloned into different vectors. In some embodiments, the generated α and β chains are incorporated into a retroviral, e.g., a lentiviral, vector.

In some embodiments, the TCR alpha and beta genes are linked via a picornavirus 2A ribosomal skip peptide so that both chains are co-expressed. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; an Hackett et al. (2010) Molecular Therapy: The Journal of the American Society of Gene Therapy. 18:674-683).

A variety of assays are known for assessing binding affinity and/or determining whether a binding molecule specifically binds to a particular ligand (e.g. peptide in the context of an MHC molecule). It is within the level of a skilled artisan to determine the binding affinity of a binding molecule, e.g., TCR, for a T cell epitope of a target polypeptide, such as by using any of a number of binding assays that are well known in the art. For example, in some embodiments, a BIAcore machine can be used to determine the binding constant of a complex between two proteins. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISAs) and radioimmunoassays (RIAs), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent), flow cytometry, sequencing and other methods for detection of expressed nucleic acids. In one example, apparent affinity for a TCR is measured by assessing binding to various concentrations of tetramers, for example, by flow cytometry using labeled tetramers. In one example, apparent KD of a TCR is measured using 2-fold dilutions of labeled tetramers at a range of concentrations, followed by determination of binding curves by non-linear regression, apparent KD being determined as the concentration of ligand that yielded half-maximal binding.

### Vectors and Methods of Engineering

The provided methods include expressing the recombinant receptors, including CARs or TCRs, for producing the genetically engineered cells expressing such binding molecules. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, nucleic acid encoding a recombinant receptor can be cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, La Jolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

Methods of lentiviral transduction are known in the art. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application Publication No. WO 2014/055668, and U.S. Pat. No. 7,446,190.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors.

Among additional nucleic acids, e.g., genes for introduction are those to improve the outcomes such as outcomes indicative of response of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### Compositions and Formulations

Also provided are populations of such cells, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy or response outcomes in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro-derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### Methods of Administration and Uses in Adoptive Cell Therapy

Provided herein are methods of administering cells, populations, and compositions described herein, and uses of such cells, populations, and compositions described herein, to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or super-type as the first subject.

In some embodiments, the subject has been treated with a therapeutic agent targeting the disease or condition, e.g., the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse.

In some aspects, the subject has not received prior treatment with another therapeutic agent.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin's lymphoma, cervical carcinoma, colorectal cancer, glioblastoma, neuroblastoma, Ewing's sarcoma, medulloblastoma, osteosarcoma, synovial sarcoma, and/or mesothelioma.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

In some embodiments, antigen associated with the disease, disorder or condition is selected from ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen.

In some embodiments, the antigen associated with the disease or disorder is selected from the group consisting of orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acetylcholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules, and/or molecules expressed by HIV, HCV, HBV or other pathogens.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺cells/kg body weight, for example, at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4⁺ and/or CD8⁺ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. For example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al J Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic outcomes are increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

### EXAMPLES

The following Examples are merely illustrative and are not intended to limit the scope or content of the invention in any way.

### Example 1— Screening of ₂RNA Candidates Against CBLB

Primary Screening of gRNAs for CBLB

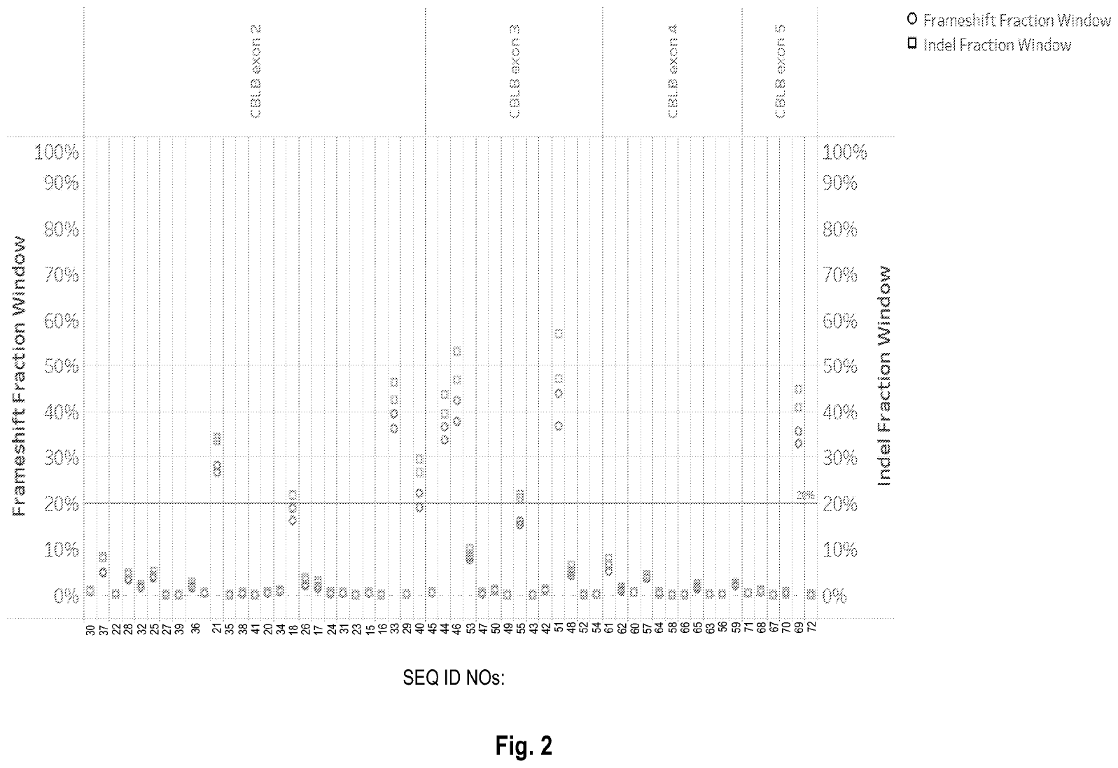

A primary screen to identify effective CBLB targeting gRNAs was conducted using 2-part Alt-R modified synthetic guides purchased from IDT. RNPs of the gRNA/Cas9 complex were nucleofected into T-cells using the Lonza 96-well Amaxa system at a concentration of 2.27 μM. T-cells were incubated for 96 hours prior to harvest and genomic DNA extraction. Indel analyses were conducted by NGS. Results are shown in FIG. 1 and FIG. 2. The sequences of the gRNAs are listed in Tables 2 and 3.

The two metrics displayed in each panel are the percentage of reads with indels (squares) and the percentage of reads with frameshift-generating indels (circles).

FIG. 1 demonstrates that there are, surprisingly, preferred hot spots for S. pyogenes gRNA targeting in the CBLB gene. FIG. 1 shows that higher than expected % indel and % frameshift mutation frequencies may be achieved when targeting the 5′ end of Exon2 or the 3′ end of Exon 4 or Exon 5. This is further exemplified by the low editing frequencies seen from the 3′ end of Exon 2 to the 5′ end of Exon 3, indicating a “dead-zone” where S. pyogenes Cas9-mediated gene-editing is inefficient.

FIG. 2 further exemplifies the surprising discovery that there are preferred hot spots for Cas9 gRNA targeting. The S. aureus gRNAs exhibit a different hot spot targeting pattern than the S. pyogenes gRNAs, with higher editing efficiencies in the 3′ end of Exon 2 to the 3′ end of Exon 3.

The results of the primary screen further demonstrate the importance of testing a panel of gRNAs to a particular target, in this case CBLB. FIGS. 1 and 2 show that many gRNAs tested unexpectedly failed to achieve reasonable editing efficiencies.

Based on the results of the primary gRNA screen, gRNAs of SEQ ID Nos: x-y were chosen to for further analysis.

Confirmation Screening of gRNAs for CBLB

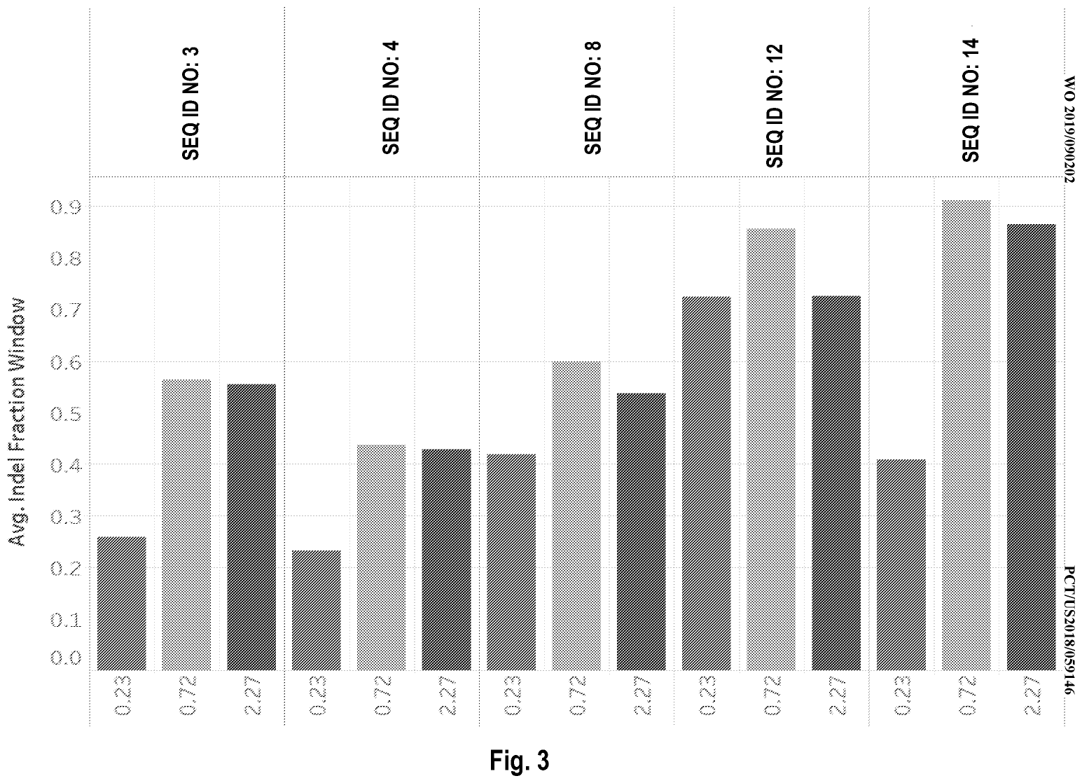

A confirmation screen was conducted with 2-part Alt-R modified synthetic gRNAs (purchased from IDT). RNPs of the gRNA/Cas9 complex were nucleofected into T-cells using the Lonza Amaxa system at a concentration of 2.27, 0.72, and 0.23 μM, respectively. T-cells were incubated for 96 hours prior to harvest and genomic DNA extraction. Indel analyses were conducted by NGS. FIG. 3 shows the average percentage of reads containing indels (indel fraction window) for the aforementioned concentrations. Rank order and relative activity was maintained for all guides compared to the primary screen. Western data (not shown) further validated knockdown of CBLB protein with all 5 guides.

Two-Part vs. Single gRNAs for CBLB

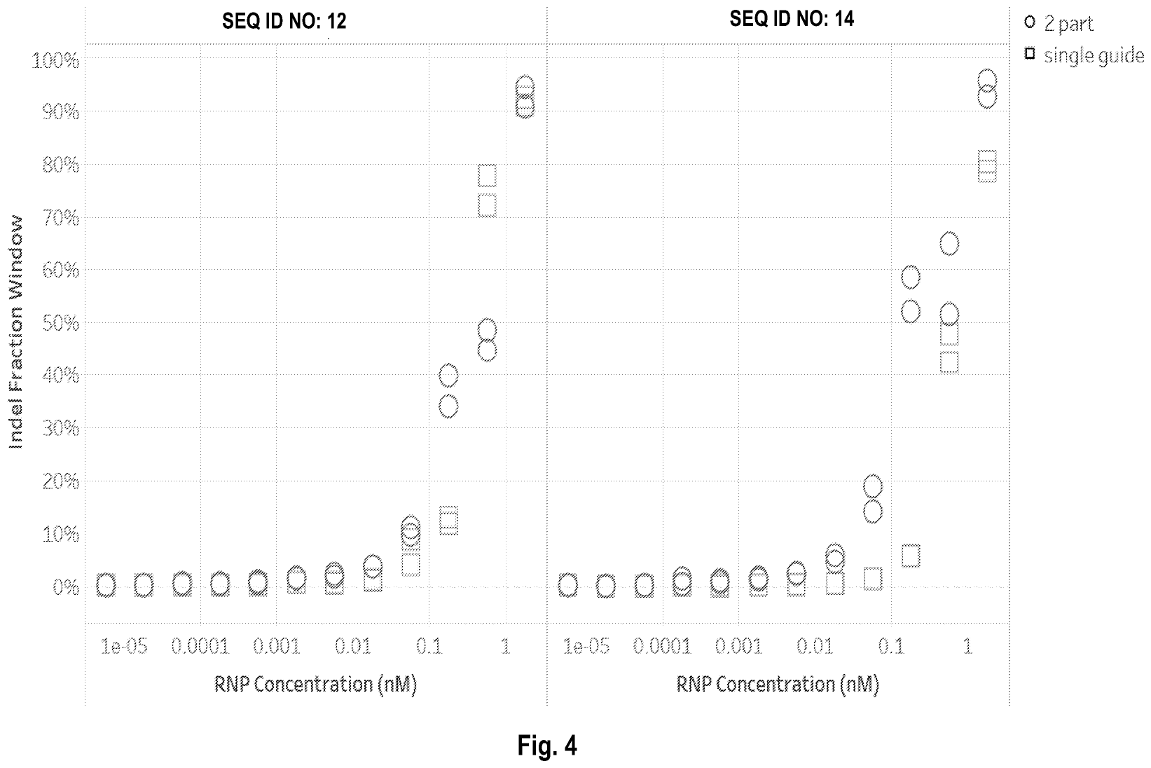

CBLB gRNAs of SEQ ID NO: X AND Y were selected as “top-tier” tools for target validation. Guides were re-ordered from IDT as unmodified sgRNAs. A bridging study to compare the original 2-part guides to the sgRNA format was conducted. RNPs were derived from the original 2-part synthetic Alt-R modified gRNAs and new unmodified sgRNAs. The resulting RNPs were run in a 12-point, semi-log dose response starting at 2 μM. T-cells were incubated for 96 hours prior to harvest and genomic DNA extraction. Indel analyses were conducted by NGS. For each panel, circles indicate the activity of the 2-part format, while squares indicate the activity of the sgRNA format at each concentration. The differences observed are within the error of the assay and the guide formats were deemed functionally equivalent. The results are shown in FIG. 4. A summary of the screening results for the preferred gRNAs is shown in Table 7.

TABLE 7
Summary of gRNAs to be used with SpCas9.

| | | Average Indel Fraction Window | | | |
|---|---|---|---|---|---|
| | Proto- | | Confir- | 2-part | |
| Guide | spacer | Primary | mation | synthetic | sgRNA |
| Name | sequence | 2.2 μM | 2.2 μM | 2.2 μM | 2.2 μM |
| SEQ ID | CGATCTGCGG | 51% | 55% | n/a | n/a |
| NO: 3 | CAGCTTGCTT | | | | |
| SEQ ID | GATCTGCGGC | 30% | 43% | n/a | n/a |
| NO: 4 | AGCTTGCTTA | | | | |
| SEQ ID | ATCTGCGGCA | 52% | 54% | n/a | n/a |
| NO: 8 | GCTTGCTTAG | | | | |
| SEQ ID | TTTCCCAATG | 66% | 73% | 93% | 92% |
| NO: 12 | GTCAATTCCA | | | | |
| SEQ ID | TAATCTGGTG | 79% | 86% | 94% | 79% |
| NO: 14 | GACCTCATGA | | | | |

In Vitro Biochemical Cutting Assay for Select gRNAs

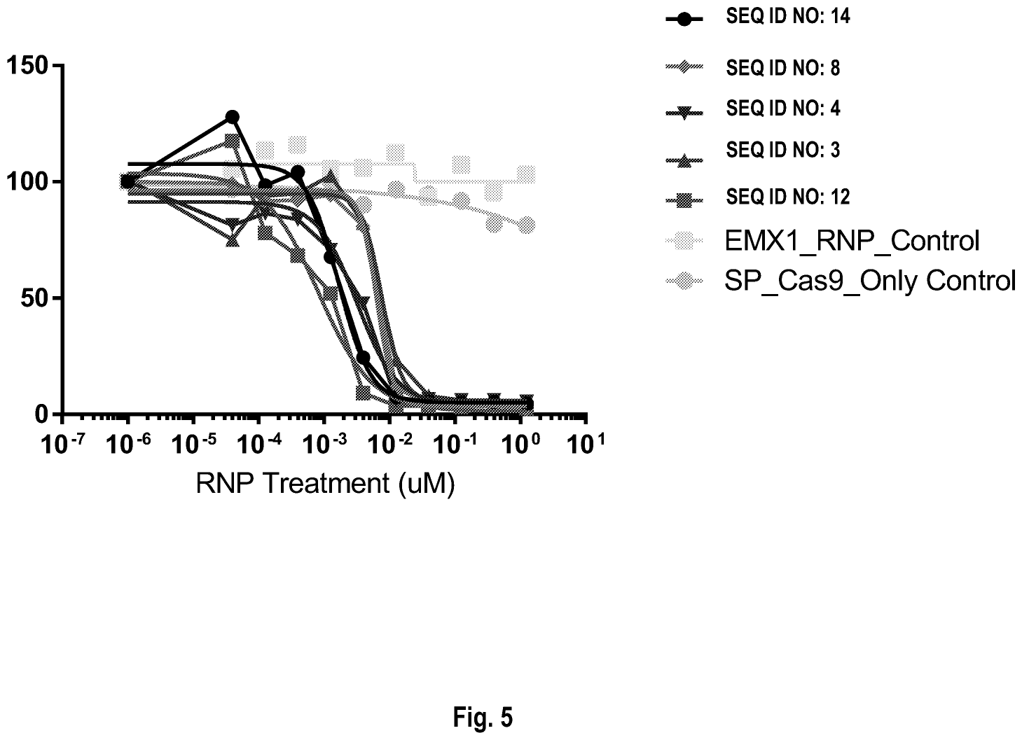

In vitro cutting assays using the preferred CBLB gRNA/Cas9 RNPs were completed on a CBLB DNA template. The results, shown in FIG. 5, demonstrate that all five gRNAs are effective at cutting CBLB in a dose-dependent manner

### Example 2 —Analysis of gRNA Candidates Against CBLB in T Cells

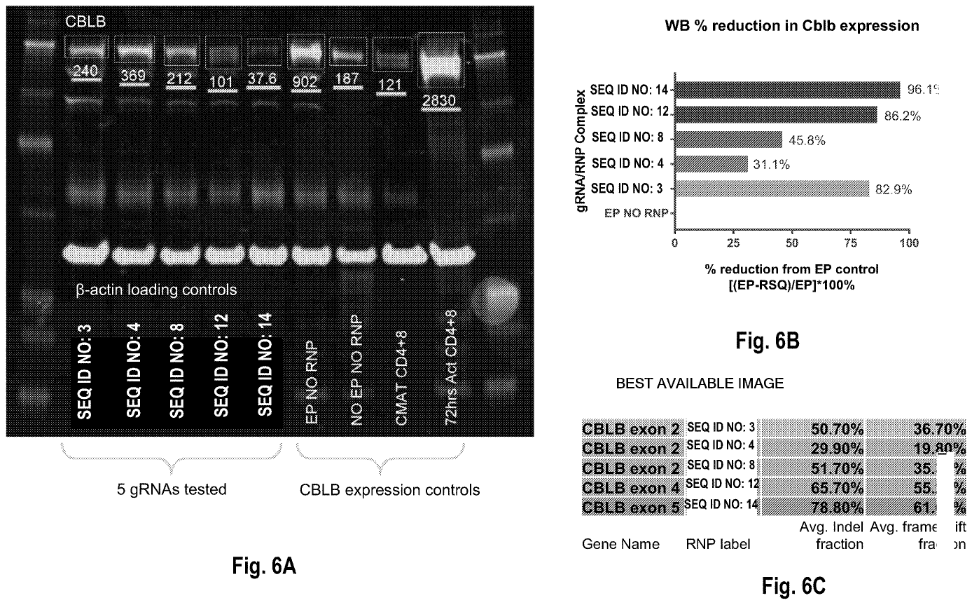

The goal of CRISPR-Cas9 editing of cells is to achieve the highest percent knock out of the target gene using the lowest possible concentration of the gRNA/Cas9 complex and the least number of off-target cutting events. To assess the 5 candidate gRNAs for CBLB gene editing, T cells were transfected with gRNA/Cas9 RNPs. Western blot was then performed to measure CBLB protein levels. FIG. 6A and 6B show that all five gRNAs are effective at reducing the expression of CBLB in T cells. The gRNA of SEQ ID NO: 14 is particularly effective.

The results of the Western blot were corroborated with NGS data for % indel and % frameshift frequencies for the five different gRNAs. All five gRNAs were effective at editing the CBLB gene, in a manner consistent with the western blot results (FIG. 6C). The gRNA of SEQ ID NO: 14 is particularly effective.

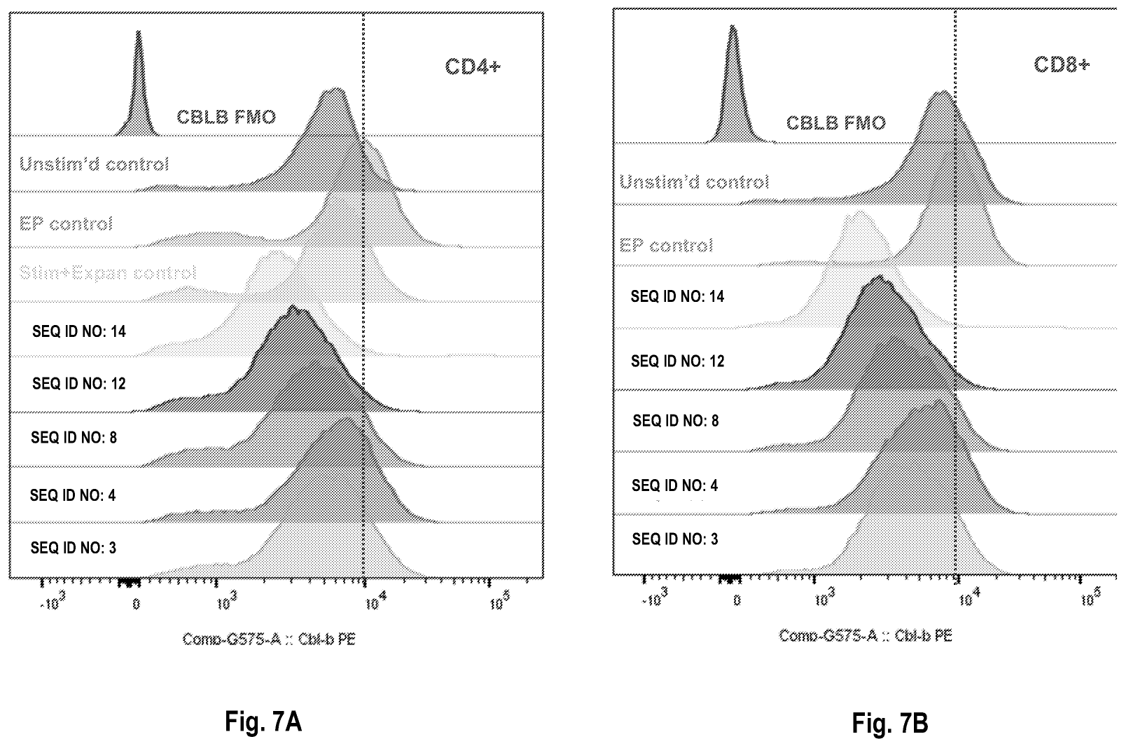

To further assess the efficiency of the five preferred gRNAs, indirect intracellular staining of CBLB in CD4+ and CD8+ T cells was performed and analyzed by FACS. Consistent with Western blot results, the five CBLB-targeting gRNAs were effective at reducing the expression of CBLB in T cells (FIG. 7A and 7B).

CBLB gRNA of SEQ ID NO: 14 was used for subsequent experiments described below.

### Example 3—In Vitro Function of Gene Editing in Primary T Cells

### T Cell Proliferation

The cell proliferative effects of CBLB gene-editing in primary T cells were determined. CD4+ and CD8+ T cells in a CBLB edited or unedited background were grown in the presence of 1.0 μg/mL plate-bound anti-CD3 antibodies. OK3T and HIT3a antibodies were used for anti-CD3 stimulation. The cells were further cultured in the absence of anti-CD28 co-stimulation and the absence of added cytokines. After growth under the various conditions, cells were then incubated with CTV for analysis by FACS.

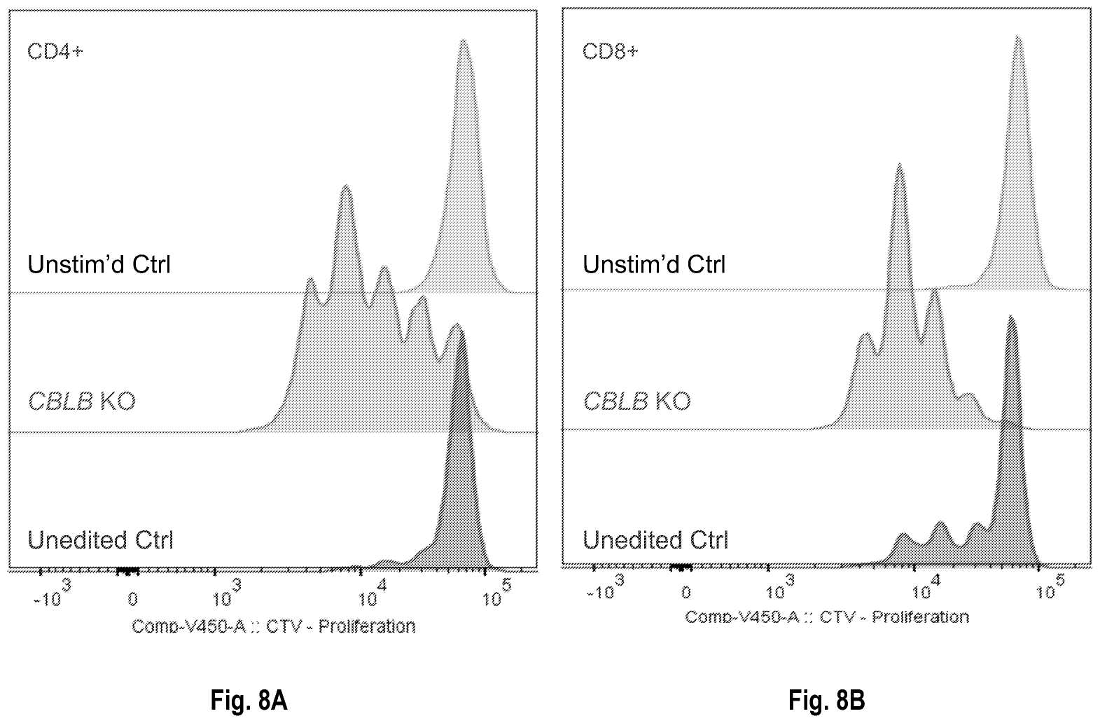

FIG. 8A and FIG. 8B show that CBLB KO CD4+ and CD8+ T cells have significantly enhanced proliferation over unedited controls in the absence of co-stimulation and the absence of added cytokines, and at sub-optimal concentrations of anti-CD3 TCR stimulation (FIG. 8A and 8B).

The cell proliferative effects of CBLB gene-editing in primary T cells were further assessed. CD4+ and CD8+ T cells in a CBLB edited or unedited background were grown in the presence of decreasing concentrations of plate-bound anti-CD3 antibodies. OK3T and HIT3a antibodies were used for anti-CD3 stimulation. The cells were further cultured in the presence of anti-CD28 co-stimulation (1.0 μg/mL anti-CD28 antibodies) and in the presence or absence of added cytokines. After growth under the various conditions, cells were then incubated with CTV for analysis by FACS.

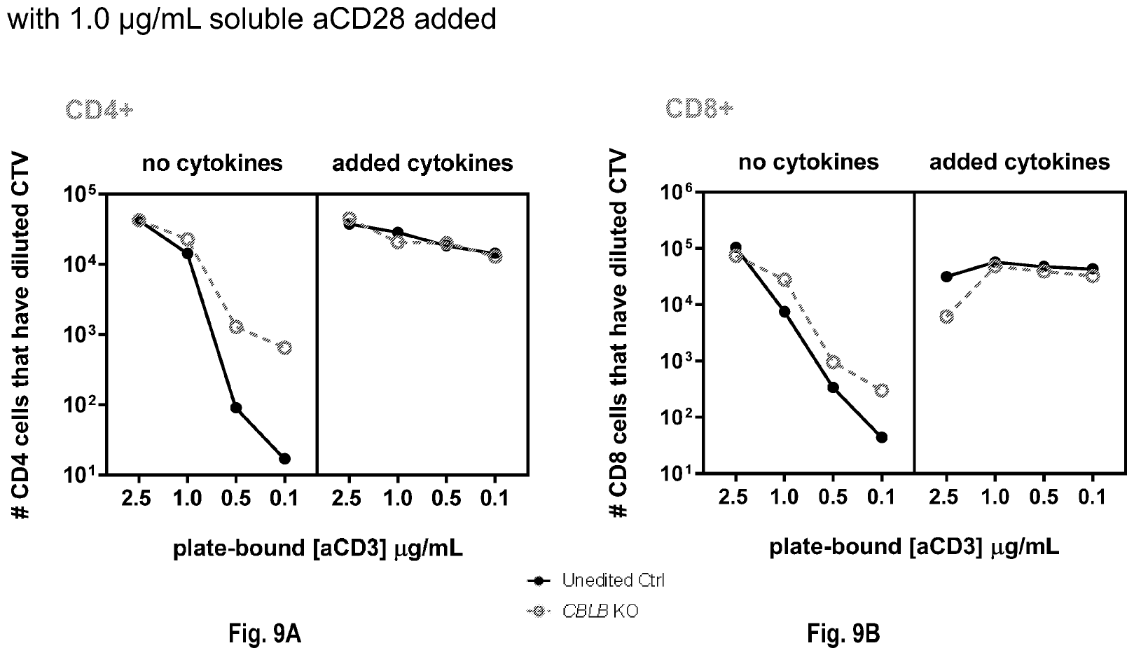

FIG. 9A and FIG. 9B show that in the presence of co-stimulation, CBLB editing increases proliferation of T cells, particularly at sub-optimal anit-CD3 stimulation. The addition of cytokines enhances proliferation regardless of whether CBLB has been edited or unedited (FIG. 9A and 9B).

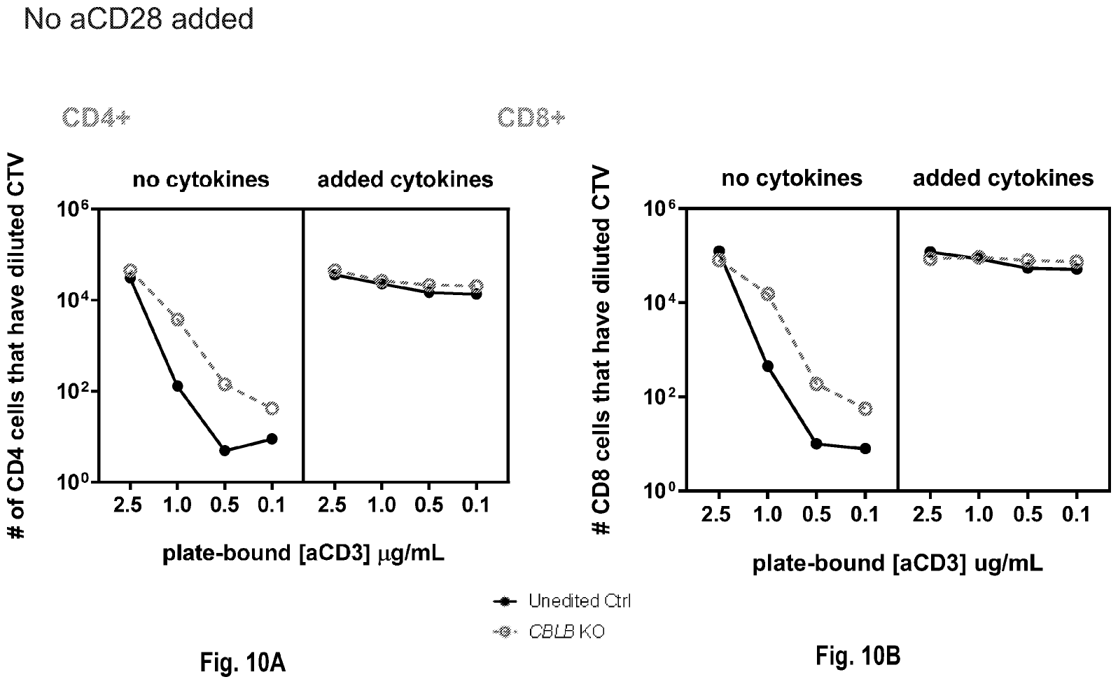

FIG. 10A and 10B show that CBLB editing has the greatest impact in the absence of anti-CD28 co-stimulation. In the absence of co-stimulation, the addition of cytokines enhances survival and proliferation regardless of whether CBLB has been edited or unedited (FIG. 10A and 10B).

### T Cell Pro-Inflammatory Cytokine Production

Pro-inflammatory cytokine production of INFγ, IL-2, and TNFα was assessed in CBLB gene-edited primary T cells. CD4+ and CD8+ T cells in a CBLB edited or unedited background were grown in the presence of decreasing amounts of plate bound anti-CD3 antibodies. OK3T antibody was used for anti-CD3 TCR stimulation. The cells were cultured in the absence of co-stimulation and the absence of added cytokines. After incubation for 20 hours, cells were then incubated in the presence of Brefeldin-A for 4 hours, and Intracellular Cytokine Staining (ICCS) was performed.

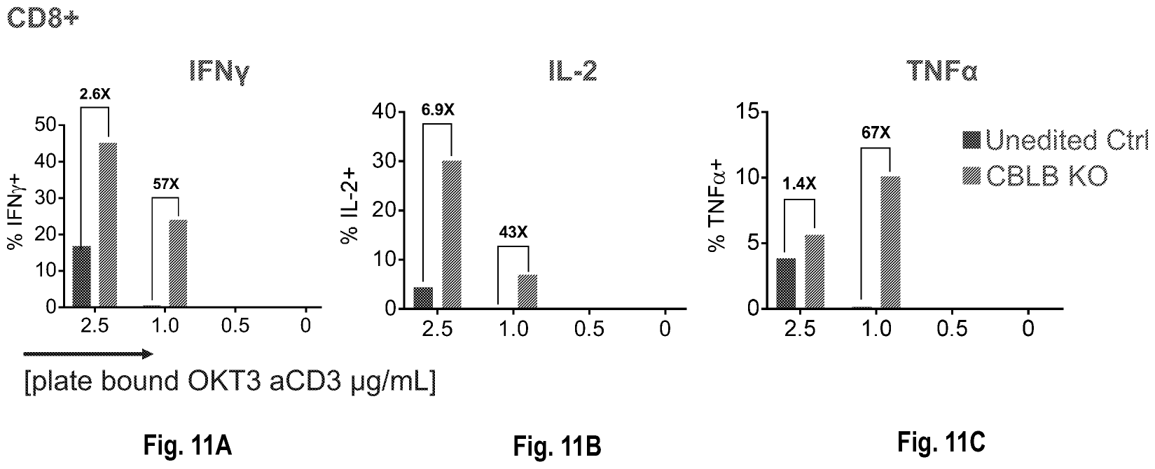

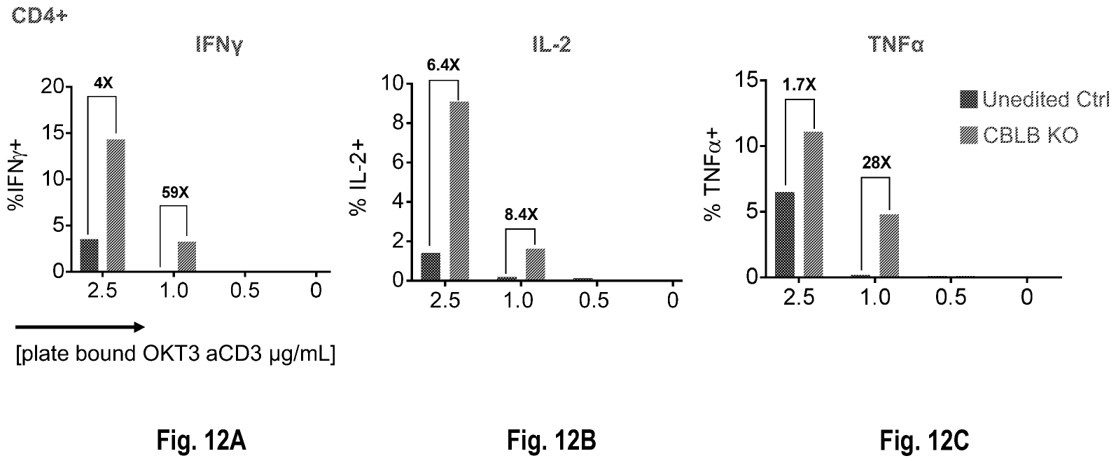

The results show that CD8+ and CD4+ CBLB KO cells have significantly enhanced production of INFγ, IL-2, and TNFa compared to unedited controls in the absence of co-stimulation and the absence of added cytokines, and at lower levels of TCR stimulation (FIG. 11A-11C and FIG. 12A-12C).

The results of Example 3 show the advantages of editing CBLB gene to produce a KO in T cells. CD4+ and CD8+ T cells can proliferate efficiently in a CBLB edited background in the absence of co-stimulation, the absence of added cytokines, and with lower levels of TCR stimulation. The results of Example 3 show that the CBLB KO cells produce higher levels of pro-inflammatory cytokines INFγ, IL-2, and TNFα than unedited controls under similar conditions

### Example 4—CBLB Gene Editing in eTCR Transduced T Cells

CBLB and eTCR Expression in CBLB Gene-Edited eTCR Transduced T Cells

Engineered TCRs (eTCRs) are designed to target an antigen of choice. Like an endogenously expressed TCR, cells engineered to express eTCRs generally require a separate co-stimulatory signal (e.g., via a receptor other than the engineered TCR receptor) for prolonged or full-blown responses following T cell activation, such as for sustained proliferation and/or target cell killing over time. The results shown are consistent with an interpretation that the provided CBLB gene-editing approaches and T cell compositions, cells and methods of treatment, are useful in providing an improved response, e.g., in the absence of costimulatory signal.

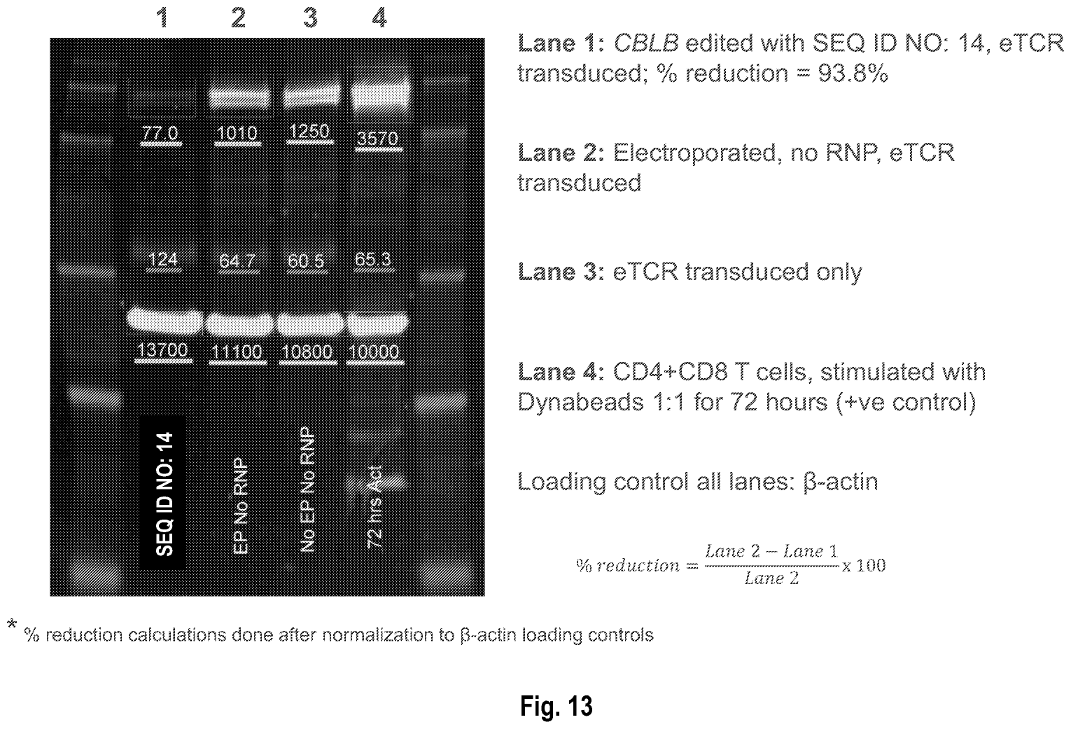

T cells were transduced with an eTCR specific for HPV E7 antigen. CBLB gene editing in HPV E7 eTCR transduced T cells was completed as previously described in the Examples above. To assess CBLB KO via gene-editing was completed in HPV E7 eTCR transduced T cells, western blot was performed to detect CBLB protein levels. As shown in FIG. 13, the gRNA-mediated CBLB KO approach was successful in HPV E7 eTCR transduced T cells. A reduction of 93.8% of CBLB was achieved.

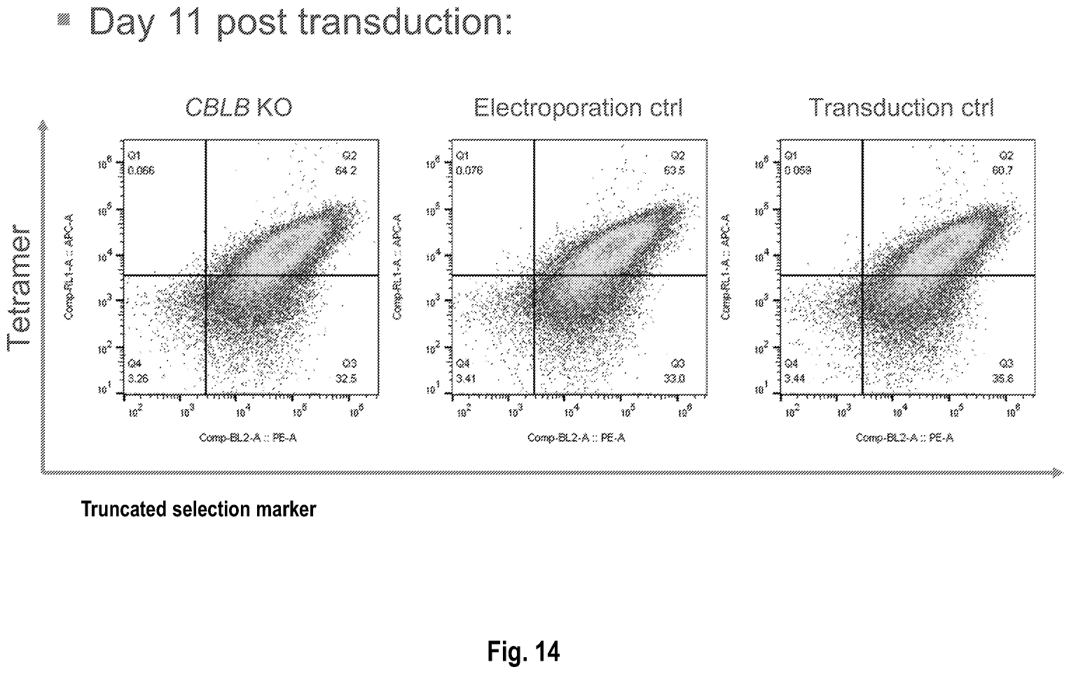

Cell surface expression of HPV E7 TCR expression (as indicated via the surrogate marker) in a CBLB KO background was assessed on day 11 post transduction. TCR expression and functional activity were assessed by flow cytometry, following staining with labeled tetramers complexed with the E7 peptide (FIG. 14).

In Vitro Function of CBLB KO HPV E7 eTCR Transduced T Cells

To assess the function of a CBLB KO in an eTCR background, an assay was carried out using an antigen presenting T2 cell line to present E7 antigen to E7 eTCR transduced T cells.

Using the T2 cell line, target cell killing activity of E7 eTCR transduced T cells, with gene-edited CBLB and unedited control, was assessed in the presence of various amounts of E7 antigen. T2 cells were pulsed overnight with decreasing concentrations of the E7 peptide. The T2 cells were then co-incubated with the E7 eTCR transduced T cells, in a CBLB KO or unedited background, at a ratio of 1:1 (E7 eTCR transduced T cells:T2 cells). The cells were further incubated in the presence or absence of CTLA4-Ig (2 μg/mL), to block co-stimulatory signal ordinarily transduced, e.g., via CD28. After a 72-hour incubation period, target cell killing and cytokine production were assessed.

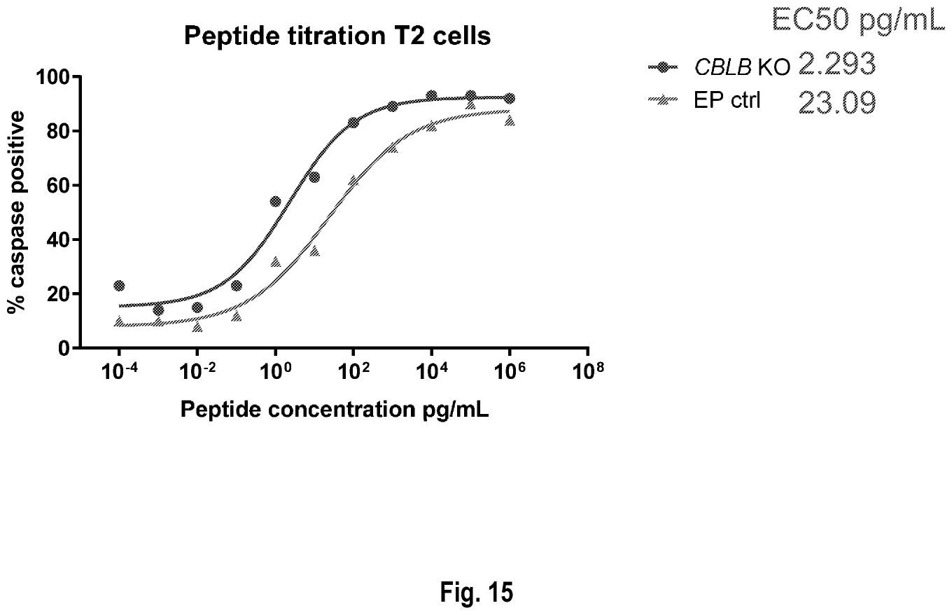

Target cell killing was assessed by measuring % caspase positive T2 cells over a range of increasing E7 peptide concentrations. The CBLB KO eTCR transduced T cells displayed superior target cell killing compared to unedited control, achieving an EC50 value (pg/ml) over ten times lower than the control (FIG. 15).

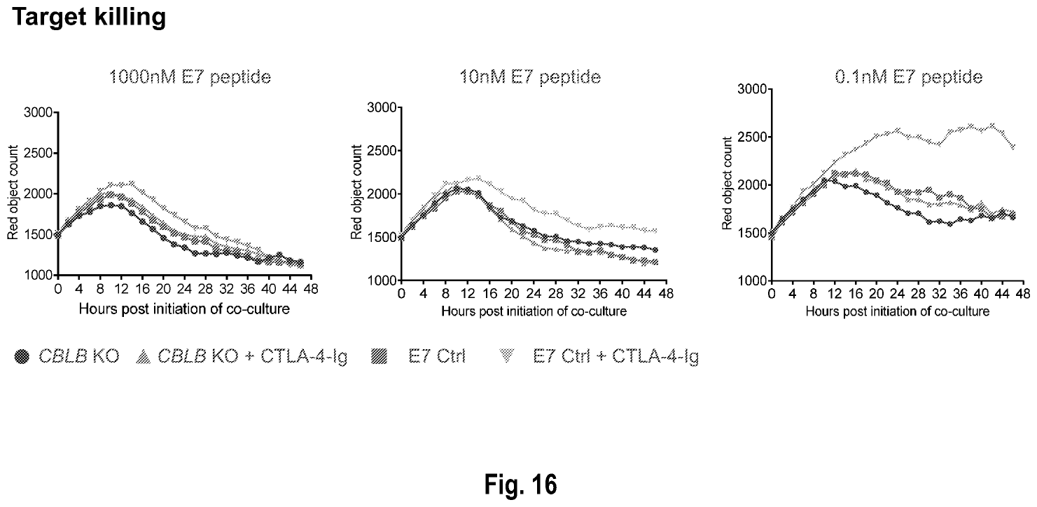

Target cell killing was also assessed using T2 cells pulsed with three concentrations of E7 peptide, 1000 nM, 10 nM, and 0.1 nM peptide. At the lowest peptide concentration, the CBLB KO eTCR transduced T cells retained the ability to kill target cells, even in the absence of co-stimulation. T2 cells continued to proliferate in the presence of the unedited control E7 eTCR transduced T cells in the absence of co-stimulation (FIG. 16).

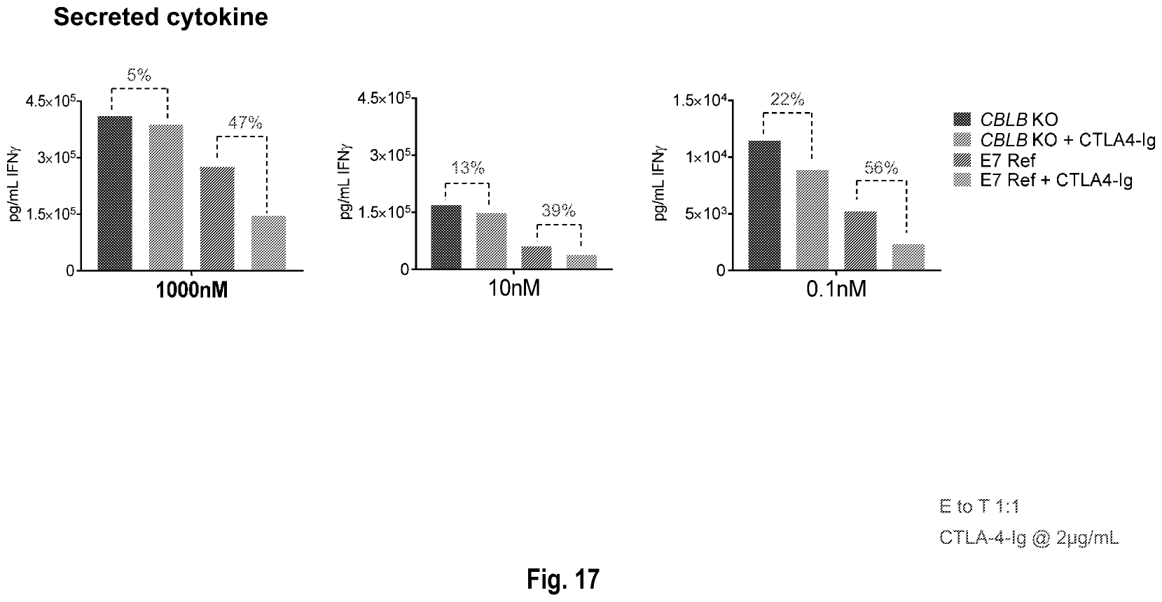

The levels of IFNγ production were also measured under the same experimental conditions as described above. CBLB KO eTCR transduced T cells exhibited higher IFNγ production levels compared to unedited E7 eTCR transduced T cells when co-stimulation was blocked with the CTLA4 reagent. The edited cells had higher levels of IFNγ secretion compared to unedited controls even when co-stimulation was not inhibited (FIG. 17).

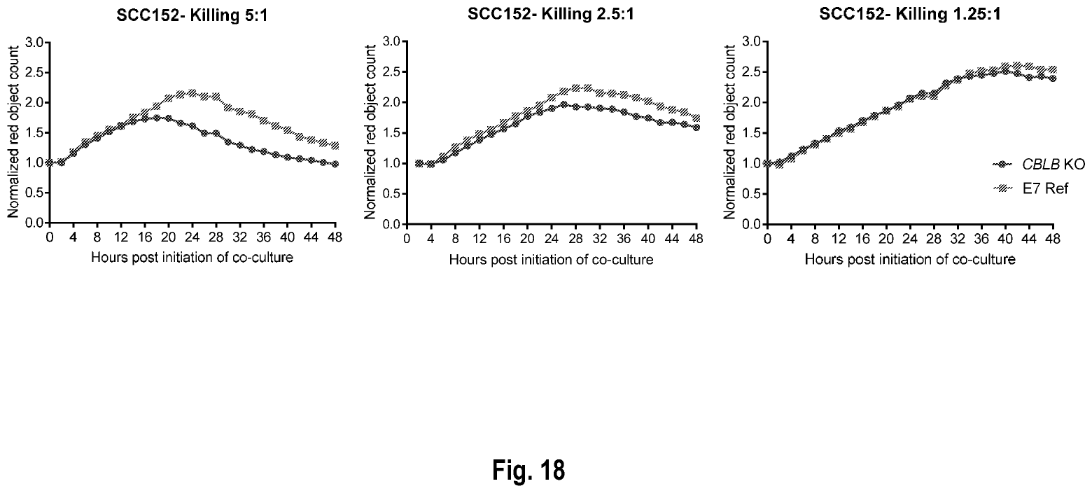

Target cell killing activity of the E7 eTCR transduced T cells was assessed using SCC157 cells, cells of an HPV transformed E7 expressing tumor cell line. In some aspects, such cell line provides constitutive E7 presentation, generally at physiological levels. Different E7 eTCR cell to SCC152 cell ratios were employed. Ratios of 5:1, 2.5:1, and 1.25:1 were used in a SCC152 killing assay, and cells were incubated for 72 hours. At the highest 5:1 ratio, CBLB KO eTCR transduced T cells exhibited higher numbers of target cell killing compared to unedited control cells (FIG. 18).

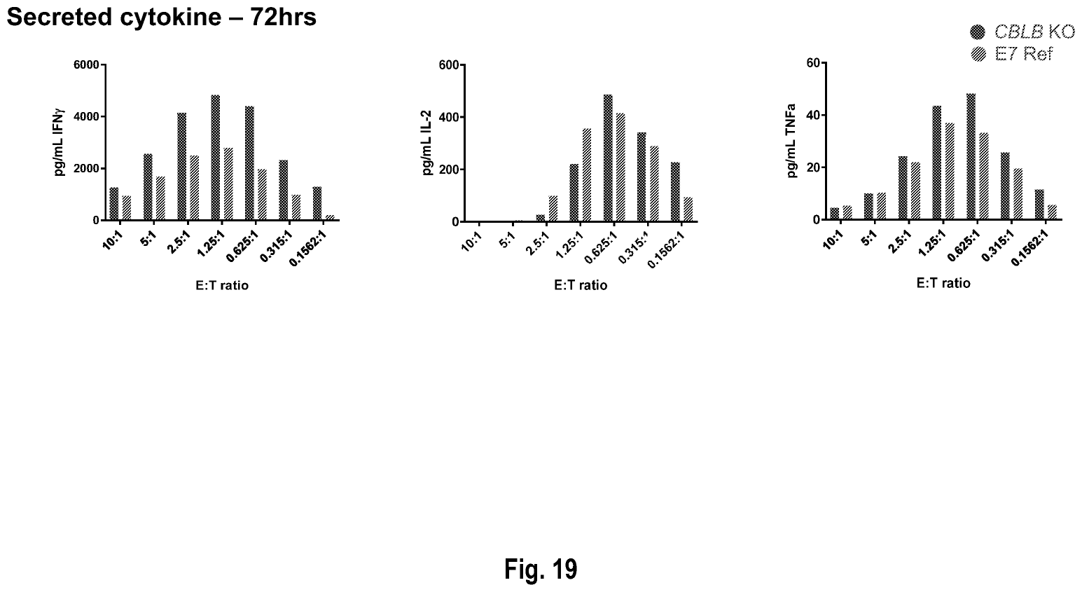

The levels of IFNγ, IL-2, and TNFa production were also measured after a 72-hour incubation at the different cell ratios described above. CBLB KO eTCR transduced cells exhibited higher levels of IFNγ and TNFα compared to unedited E7 eTCR transduced T cells at all ratios tested. CBLB KO eTCR transduced cells exhibited higher levels of IL-2 at cell ratios of 0.625:1 or lower (FIG. 19).

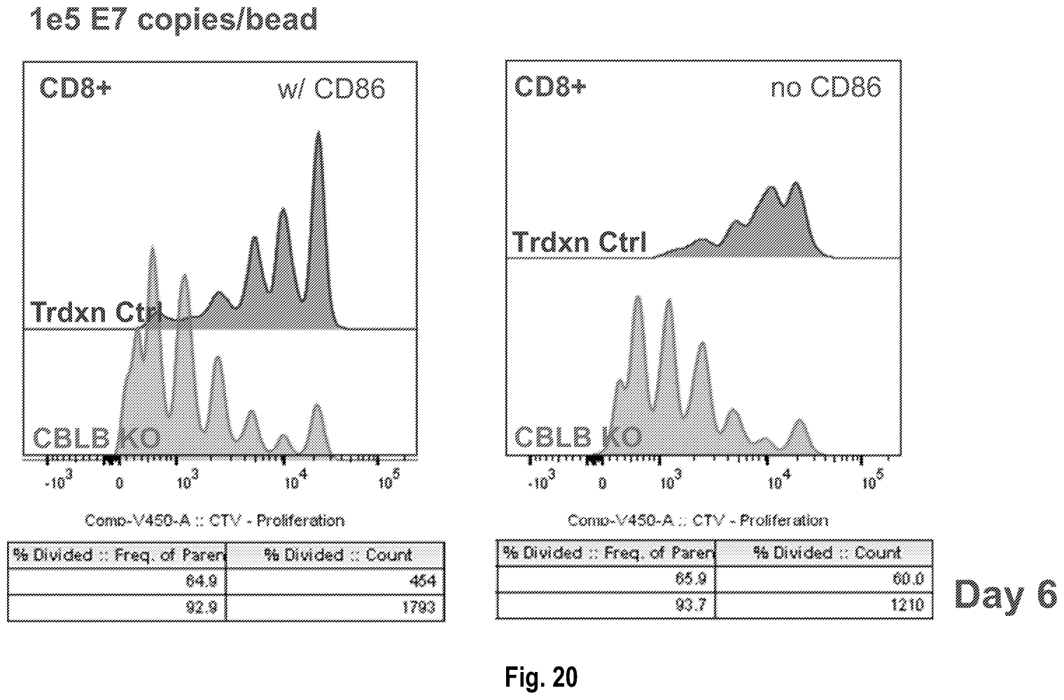

E7 conjugated beads were utilized to assess proliferation following antigen binding of E7 eTCR transduced T cells in a CBLB KO background. 5×10⁴ beads conjugated with E7:MHC1 monomers, with and without CD86, were used in the assay. The beads were incubated with cells and labeled with CTV. Cells were harvested at 6 days to assess viability and proliferation. Viability was determined by FACS analysis and it was observed that bead co-culturing lead to a decrease in viability in both the CBLB edited (data not shown) and unedited cells (data not shown). As shown in FIG. 20, at day 6, the CBLB KO E7 eTCR transduced T cells showed greater proliferation compared to the unedited control cells (FIG. 20). This effect was observed both with and without CD86 co-stimulation.

The collective results of Example 4 are consistent with the utility of CBLB gene-editing methods in connection with eTCR-transduced T cells such as for adoptive cell therapy, and of eTCR-transduced T cells for adoptive cell therapy in which CBLB expression or gene is reduced or disrupted, such as CBLB knockout. Unlike second-generation CAR T cells, in which the CAR generally possess a built-in co-stimulatory domain in addition to a domain capable of delivering a primary signal (such as a CD3zeta domain), which along with the primary signaling domain can be triggered in response to a single antigen-binding event, eTCR cells typically must receive co-stimulatory signals via a mechanism or receptor separate from the antigen-binding TCR, such as via a signaling domain present in a separate receptor, such as CD28 (e.g., by binding through B7.1 or B7.2). The results are consistent with a conclusion that knocking out CBLB in an engineered TCR transduced T cell background can improve function following antigen-driven stimulation, even in the absence of a separate costimulatory receptor.

As shown in Example 4, the CBLB KO eTCR transduced T cells generally were more sensitive to antigenic stimulation, they exhibited increased target cell killing, increased production of pro-inflammatory cytokines, and exhibited increased proliferation at lower antigen densities compared to unedited controls, all in the absence of co-stimulation. These characteristics may be desirable in the therapeutic context. Low tumor antigen densities and a weakened co-stimulatory environment can represent considerable barriers to treating tumors in certain contexts. The provided cells and methods may be useful for the production of cells for administration in the clinic.

### Example 5—CBLB KO/eTCR Transduced Cells in an In Vivo Tumor Model

Persistence was assessed in an in vivo tumor model bearing SCC152 tumors.

The tumor xenograft mouse model was generated by implanting nod scid gamma (NSG) mice with tumor cells derived from the head and neck squamous cell carcinoma 152 (SCC152) cell line via subcutaneous injection. On day 31 post initial tumor cell implantation, cryopreserved CBLB KO eTCR transduced T cells and unedited electroporated (EP) and unedited unelectroporated eTCR transduced T cells were thawed and resuspended, and infused into recipient mice in the following groups and concentrations:

| | # of T cells infused |
|---|---|
| Group | per mouse |
| CBLB KO eTCR transduced | 2 × 10⁶ |
| CBLB KO eTCR transduced | 1 × 10⁶ |
| Unedited electroporated eTCR transduced | 2 × 10⁶ |
| control | |
| Unedited unelectroporated eTCR transduced | 2 × 10⁶ |
| control | |
| Tumor only control | n/a |

Tumors are measured in all mice every 5 days by Biopticon Tumor Imager tumor scanning device for the first week post T cell infusion and every 3-5 days thereafter. Blood is drawn every 7 days for 4 weeks total. Drawn blood is stained using T cell lineage identifying fluorophore conjugated antibodies and an E7-specific tetramer to assess the frequency of E7 specific T cells via flow cytometry.

In some embodiments, reduced tumor growth, tumor growth arrest and/or tumor clearance in mice infused with CBLB KO eTCR transduced T cells, as compared to unedited and tumor only controls are observed.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1. A genome editing system comprising:
a guide RNA (gRNA) comprising a targeting domain that is complementary with a target sequence of a Casitas B-lineage lymphoma proto-oncogene-b (CBLB) gene; and
an RNA-guided nuclease.

2. The genome editing system of claim 1, wherein:
the target sequence of the CBLB gene comprises the sequence of exon 2, exon 4, or exon 5;
the target sequence of the CBLB gene comprises the sequence selected from the group consisting of SEQ ID NOs: 88-92;
the targeting domain has at least 85% complementarity to the target sequence of the CBLB gene;
the targeting domain is configured to form a double strand break or a single strand break within about 500 bp, about 450 bp, about 400 bp, about 350 bp, about 300 bp, about 250 bp, about 200 bp, about 150 bp, about 100 bp, about 50 bp, about 25 bp, or about 10 bp of an CBLB target position, thereby altering CBLB gene expression, optionally wherein CBLB gene expression is knocked out or knocked down;
the targeting domain is configured to target a coding region or a non-coding region of said CBLB gene, wherein said non-coding region comprises a promoter region, an enhancer region, an intron, a 3′ UTR, a 5′ UTR, or a polyadenylation signal region of said CBLB gene, optionally wherein the coding region is selected from exon 2, exon 4, and exon 5; and/or
the targeting domain comprises a nucleotide sequence that is identical to, or differs by no more than 3 nucleotides from, a nucleotide sequence selected from the group consisting of SEQ ID NOs: 1 to 14.

3-9. (canceled)

10. The genome editing system of claim 1, wherein said RNA-guided nuclease is an S. pyogenes Cas9 nuclease, optionally the S. pyogenes Cas9 nuclease recognizes a Protospacer Adjacent Motif (PAM) of NGG, and said targeting domain comprises a nucleotide sequence that is identical to, or differs by no more than about 3 nucleotides from, a nucleotide sequence selected from the group consisting of:
(a) SEQ ID NO: 3;
(b) SEQ ID NO: 4;
(c) SEQ ID NO: 8;
(d) SEQ ID NO: 12; and
(e) SEQ ID NO: 14.

11. (canceled)

12. The genome editing system of claim 1, wherein:
the RNA-guided nuclease is an S. aureus Cas9 nuclease, optionally wherein the S. aureus Cas9 nuclease recognizes a PAM of either NNNRRT or NNNRRV; and/or
the RNA-guided nuclease is a mutant Cas9 nuclease.

13-14. (canceled)

15. The genome editing system of claim 1, wherein:
the gRNA is a modular gRNA or a chimeric gRNA;
the targeting domain has a length of about 16, about 17, about 18, about 19, about 20, about 21, about 22, about 23, about 24, about 25, or about 26 nucleotides;
the targeting domain comprises at least about 18 contiguous nucleotides that are complementary to the CBLB gene; and/or
the genome editing system comprises two, three or four distinct gRNAs.

16-18. (canceled)

19. The genome editing system of claim 1 for use in reducing or eliminating CBLB gene expression in a cell, optionally wherein:
the cell is from a subject suffering from cancer;
expression of CBLB is reduced by 30% or more relative to a baseline measurement, optionally wherein expression of CBLB protein is determined by Western blot or indirect intracellular staining flow cytometry; and/or
a frame-shift mutation is introduced into the CBLB gene.

20-51. (canceled)

52. A method of altering CBLB gene expression in a cell, comprising administering to said cell
the genome editing system of claim 1, or
a vector comprising a polynucleotide encoding the gRNA and a polynucleotide encoding the RNA-guided nuclease.

53. The method of claim 52, wherein:
CBLB gene expression is knocked out or knocked down;
the cell is from a subject suffering from cancer; and/or
the gRNA and the RNA-guided nuclease comprise a ribonucleoprotein (RNP) complex, optionally wherein:
the method comprises administering two or more RNP complexes comprising distinct gRNAs;
the RNP complex comprise enzymatically active Cas9 (eaCas9) nucleases;
the RNP complex comprise eaCas9 nucleases that form double strand breaks in a target nucleic acid or form single strand breaks in a target nucleic acid; and/or
two RNP complexes comprising distinct gRNAs are used to form offset single strand breaks in the CBLB gene in the cell.

54-59. (canceled)

60. A cell comprising the genome editing system of claim 1, optionally wherein:
the cell expresses CBLB;
the cell is a T cell or a Natural Killer (NK) cell, optionally further comprising an engineered T Cell Receptor (eTCR), a Chimeric Antigen Receptor (CAR), or a recombinant or engineered antigen receptor.

61-63. (canceled)

64. A cell altered according to the method of claim 52.

65. The method of claim 52, comprising:
a) contacting the cell with a sufficient amount of the gRNA that targets CBLB and the RNA-guided nuclease; and
b) forming a first DNA double strand break at or near a CBLB target position in a CBLB gene of the cell, wherein the first DNA double strand break is repaired by NHEJ, wherein said repair alters the expression of the CBLB gene, optionally further comprising forming a second DNA double strand break at or near the CBLB target position.

66-67. (canceled)

68. The method of claim 65, wherein:
the first and second double strand breaks are formed within about 500 bp, about 450 bp, about 400 bp, about 350 bp, about 300 bp, about 250 bp, about 200 bp, about 150 bp, about 100 bp, about 50 bp, about 25 bp, or about 10 bp of a CBLB target position;
the first and second double strand breaks are formed in a coding region or a non-coding region of said CBLB gene, wherein said non-coding region comprises a promoter region, an enhancer region, an intron, a 3′ UTR, a 5′ UTR, or a polyadenylation signal region of said CBLB gene, optionally wherein the coding region is selected from exon 2, exon 4, and exon 5;
the targeting domain comprises a nucleotide sequence that is identical to, or differs by no more than about 3 nucleotides from, a nucleotide sequence selected from the group consisting of SEQ ID NOS: 1 to 14; and/or
the RNA-guided nuclease is an S. pyogenes Cas9 nuclease, and said targeting domain comprises a nucleotide sequence that is identical to, or differs by no more than about 3 nucleotides from, a nucleotide sequence selected from the group consisting of:
(a) SEQ ID NO: 3;
(b) SEQ ID NO: 4;
(c) SEQ ID NO: 8;
(d) SEQ ID NO: 12; and
(e) SEQ ID NO: 14.

69-75. (canceled)

76. The method of claim 65, wherein the NHEJ repair produces an insertion or deletion with a frequency of greater than or equal to 20%, 30%, 40%, or 50%.

77-84. (canceled)

85. A method of treating cancer in subject, comprising administering to the subject engineered immune cells, wherein the engineered immune cells have reduced expression of a CBLB gene, and optionally an engineered T Cell Receptor (eTCR) or a Chimeric Antigen Receptor (CAR), wherein the engineered immune cells have an insertion or a deletion near the CBLB gene.

86. The method of claim 85, wherein:
the engineered immune cells comprise T cells or NK cells, optionally wherein the T cells are CD4+ T cells and/or CD8+ T cells;
the eTCR or CAR has antigen specificity to a cancer cell;
CBLB expression in the engineered immune cells is reduced by introducing into the immune cells a genome editing system comprising a gRNA comprising a targeting domain that is complementary with a target sequence of said CBLB gene, and a RNA-guided nuclease;
the engineered immune cells maintain or have enhanced proliferation in the absence of CD28 co-stimulation relative to a non-engineered immune cell;
the engineered immune cells maintain or have enhanced proliferation in the absence of cytokines relative to non-engineered immune cells;
the engineered immune cells maintain or have increased expression of IFN-gamma, IL-2, and TNF-alpha relative to non-engineered immune cells; and/or
the engineered immune cells maintain or have increased target cell killing capacity relative to non-engineered immune cells.

87-93. (canceled)

94. A method of enhancing the proliferation of immune cells in which CD28 co-stimulation is reduced or absent, comprising introducing into the immune cells a genome editing system comprising a gRNA molecule comprising a targeting domain that is complementary with a target sequence of said CBLB gene, and a RNA-guided nuclease, and reducing CBLB expression in the immune cells.

95. The method of claim 94, further comprising enhancing proliferation in the absence or reduction of cytokines, optionally wherein there is an absence or reduction of the cytokines IL-2, IL-7, and IL-15.

96. (canceled)

97. A composition comprising a plurality of engineered T cells, wherein said engineered T cells exhibit reduced CBLB gene expression relative to non-engineered T cells.

98. The composition of claim 97, wherein:
the engineered T cells exhibit a CBLB gene expression level that is about 50%, about 40%, about 30%, about 20%, about 10% or about 5% the level of CBLB expression in non-engineered T cells;
the engineered T cells further comprise expression of an eTCR or a CAR;
the T cells are CD4+ T cells and/or CD8+ T cells; and/or
the engineered T cells are further characterized by possessing one or more of:
a) maintained or increased proliferation in the absence of CD28 co-stimulation;
b) maintained or increased target cell killing in the absence of CD28 co-stimulation;
c) greater sensitivity to a target antigen;
d) maintained or increased target cell killing in the presence of reduced target antigen; and
e) an increased ability to produce cytokines.

99-101. (canceled)

102. The composition of claim 97, wherein the engineered T cells are produced by contacting non-engineered T cells with a genome editing system comprising:
a gRNA comprising a targeting domain that is complementary with a target sequence of a CBLB gene; and
an RNA-guided nuclease.

103. The composition of claim 102, wherein:
the engineered T cells further comprise a vector or a polynucleotide that expresses an eTCR or a CAR, optionally wherein the vector is a viral vector and/or wherein the polynucleotide is integrated into the genome of the T cell; and/or
the RNA-guided nuclease is an S. pyogenes Cas9 nuclease, and said targeting domain comprises a nucleotide sequence that is identical to, or differs by no more than about 3 nucleotides from, a nucleotide sequence selected from the group consisting of:
(a) SEQ ID NO: 3;
(b) SEQ ID NO: 4;
(c) SEQ ID NO: 8;
(d) SEQ ID NO: 12; and
(e) SEQ ID NO: 14.

104-107. (canceled)

108. A composition comprising:
a first gRNA that targets a CBLB gene;
a second gRNA that targets a TRAC gene; and
a third gRNA that targets a TRBC gene;
optionally further comprising an RNA-guided nuclease.

109. A method of treating cancer in a subject, comprising administering to the subject a plurality of the engineered immune cell of claim 114.

110. The method of claim 109, wherein the engineered immune cells express an engineered T Cell Receptor (eTCR) or a Chimeric Antigen Receptor (CAR), optionally wherein the eTCR or CAR has specificity to a cancer antigen.

111-113. (canceled)

114. An engineered immune cell comprising:
a CBLB gene knockout or knockdown;
a TRAC gene knockout or knockdown; and
a TRBC gene knockout or knockdown.

115. A method of producing an engineered immune cell having an insertion or deletion disrupting a CBLB gene, a TRAC gene, and a TRBC gene, comprising:
i) isolating an immune cell; and
ii) contacting the immune cell with the genome editing system of claim 107 to generate an engineered immune cell, optionally wherein the cells further comprise an engineered T Cell Receptor (eTCR) or a Chimeric Antigen Receptor (CAR).

116-119. (canceled)


SEQUENCE LISTING (98 sequences)

SEQ ID NO: 1 - DNA, 20 bp, Artificial Sequence
  source: /note=“Description of Artificial Sequence Synthetic oligonucleotide”
agcaagctgccgcagatcgc

SEQ ID NO: 2 - DNA, 20 bp, Artificial Sequence
  source: /note=“Description of Artificial Sequence Synthetic oligonucleotide”
tacccaaaattcgacctttt

SEQ ID NO: 3 - DNA, 20 bp, Artificial Sequence
  source: /note=“Description of Artificial Sequence Synthetic oligonucleotide”
cgatctgcggcagcttgctt

SEQ ID NO: 4 - DNA, 20 bp, Artificial Sequence
  source: /note=“Description of Artificial Sequence Synthetic oligonucleotide”
gatctgcggcagcttgctta

SEQ ID NO: 5 - DNA, 20 bp, Artificial Sequence
  source: /note=“Description of Artificial Sequence Synthetic oligonucleotide”
ggcagaaaccctggtggtcg

SEQ ID NO: 6 - DNA, 20 bp, Artificial Sequence
  source: /note=“Description of Artificial Sequence Synthetic oligonucleotide”
ggatttcctcctcgaccacc

SEQ ID NO: 7 - DNA, 20 bp, Artificial Sequence
  source: /note=“Description of Artificial Sequence Synthetic oligonucleotide”
gggtattattgatgctattc

SEQ ID NO: 8 - DNA, 20 bp, Artificial Sequence
  source: /note=“Description of Artificial Sequence Synthetic oligonucleotide”
atctgcggcagcttgcttag

SEQ ID NO: 9 - DNA, 20 bp, Artificial Sequence
  source: /note=“Description of Artificial Sequence Synthetic oligonucleotide”
cgtaaatgctgatatgtatc

SEQ ID NO: 10 - DNA, 20 bp, Artificial Sequence
  source: /note=“Description of Artificial Sequence Synthetic oligonucleotide”
cccgttttgactttttcata

SEQ ID NO: 11 - DNA, 20 bp, Artificial Sequence
  source: /note=“Description of Artificial Sequence Synthetic oligonucleotide”
tgatacgaaagttatctccc

SEQ ID NO: 12 - DNA, 20 bp, Artificial Sequence
  source: /note=“Description of Artificial Sequence Synthetic oligonucleotide”
tttcccaatggtcaattcca

SEQ ID NO: 13 - DNA, 20 bp, Artificial Sequence
  source: /note=“Description of Artificial Sequence Synthetic oligonucleotide”
ccaccagattagctctggcc

SEQ ID NO: 14 - DNA, 20 bp, Artificial Sequence
  source: /note=“Description of Artificial Sequence Synthetic oligonucleotide”
taatctggtggacctcatga

SEQ ID NO: 15 - DNA, 21 bp, Artificial Sequence
  source: /note=“Description of Artificial Sequence Synthetic oligonucleotide”
tggcagaaaccctggtggtcg

SEQ ID NO: 16 - DNA, 21 bp, Artificial Sequence
  source: /note=“Description of Artificial Sequence Synthetic oligonucleotide”
atggcagaaaccctggtggtc

SEQ ID NO: 17 - DNA, 21 bp, Artificial Sequence
  source: /note=“Description of Artificial Sequence Synthetic oligonucleotide”
ggggatttcctcctcgaccac

SEQ ID NO: 18 - DNA, 21 bp, Artificial Sequence
  source: /note=“Description of Artificial Sequence Synthetic oligonucleotide”
aatccccgaaaaggtcgaatt

SEQ ID NO: 19 - DNA, 21 bp, Artificial Sequence
  source: /note=“Description of Artificial Sequence Synthetic oligonucleotide”
cgatctgcggcagcttgctta

SEQ ID NO: 20 - DNA, 21 bp, Artificial Sequence
  source: /note=“Description of Artificial Sequence Synthetic oligonucleotide”
atacccaaaattcgacctttt

SEQ ID NO: 21 - DNA, 21 bp, Artificial Sequence
  source: /note=“Description of Artificial Sequence Synthetic oligonucleotide”
cttagggggtccaactgcatc

SEQ ID NO: 22 - DNA, 21 bp, Artificial Sequence
  source: /note=“Description of Artificial Sequence Synthetic oligonucleotide”
tcgcaggaccgtggagaagac

SEQ ID NO: 23 - DNA, 21 bp, Artificial Sequence
  source: /note=“Description of Artificial Sequence Synthetic oligonucleotide”
gcagaaaccctggtggtcgag

SEQ ID NO: 24 - DNA, 21 bp, Artificial Sequence
  source: /note=“Description of Artificial Sequence Synthetic oligonucleotide”
tggtggtcgaggaggaaatcc

SEQ ID NO: 25 - DNA, 21 bp, Artificial Sequence
  source: /note=“Description of Artificial Sequence Synthetic oligonucleotide”
gctgccgcagatcgcaggacc

SEQ ID NO: 26 - DNA, 21 bp, Artificial Sequence
  source: /note=“Description of Artificial Sequence Synthetic oligonucleotide”
aggaggaaatccccgaaaagg

SEQ ID NO: 27 - DNA, 21 bp, Artificial Sequence
  source: /note=“Description of Artificial Sequence Synthetic oligonucleotide”
tgcgatctgcggcagcttgct

SEQ ID NO: 28 - DNA, 21 bp, Artificial Sequence
  source: /note=“Description of Artificial Sequence Synthetic oligonucleotide”
gccgcagatcgcaggaccgtg

SEQ ID NO: 29 - DNA, 21 bp, Artificial Sequence
  source: /note=“Description of Artificial Sequence Synthetic oligonucleotide”
gccattcattgagtttgccat

SEQ ID NO: 30 - DNA, 21 bp, Artificial Sequence
  source: /note=“Description of Artificial Sequence Synthetic oligonucleotide”
gtggagaagacttggaagctc

SEQ ID NO: 31 - DNA, 21 bp, Artificial Sequence
  source: /note=“Description of Artificial Sequence Synthetic oligonucleotide”
cagaaaccctggtggtcgagg

SEQ ID NO: 32 - DNA, 21 bp, Artificial Sequence
  source: /note=“Description of Artificial Sequence Synthetic oligonucleotide”
ctgccgcagatcgcaggaccg

SEQ ID NO: 33 - DNA, 21 bp, Artificial Sequence
  source: /note=“Description of Artificial Sequence Synthetic oligonucleotide”
caccagggtttctgccattca

SEQ ID NO: 34 - DNA, 21 bp, Artificial Sequence
  source: /note=“Description of Artificial Sequence Synthetic oligonucleotide”
tacccaaaattcgaccttttc

SEQ ID NO: 35 - DNA, 21 bp, Artificial Sequence
  source: /note=“Description of Artificial Sequence Synthetic oligonucleotide”
ttgatgctattcaggatgcag

SEQ ID NO: 36 - DNA, 21 bp, Artificial Sequence
  source: /note=“Description of Artificial Sequence Synthetic oligonucleotide”
taagcaagctgccgcagatcg

SEQ ID NO: 37 - DNA, 21 bp, Artificial Sequence
  source: /note=“Description of Artificial Sequence Synthetic oligonucleotide”
cgcaggaccgtggagaagact

SEQ ID NO: 38 - DNA, 21 bp, Artificial Sequence
  source: /note=“Description of Artificial Sequence Synthetic oligonucleotide”
ttgggtattattgatgctatt

SEQ ID NO: 39 - DNA, 21 bp, Artificial Sequence
  source: /note=“Description of Artificial Sequence Synthetic oligonucleotide”
gcgatctgcggcagcttgctt

SEQ ID NO: 40 - DNA, 21 bp, Artificial Sequence
  source: /note=“Description of Artificial Sequence Synthetic oligonucleotide”
aaattccagatggcaaactca

SEQ ID NO: 41 - DNA, 21 bp, Artificial Sequence
  source: /note=“Description of Artificial Sequence Synthetic oligonucleotide”
aatacccaaaattcgaccttt

SEQ ID NO: 42 - DNA, 21 bp, Artificial Sequence
  source: /note=“Description of Artificial Sequence Synthetic oligonucleotide”
aacttgcccaactcagtgaga

SEQ ID NO: 43 - DNA, 21 bp, Artificial Sequence
  source: /note=“Description of Artificial Sequence Synthetic oligonucleotide”
aaagtactcattctcactgag

SEQ ID NO: 44 - DNA, 21 bp, Artificial Sequence
  source: /note=“Description of Artificial Sequence Synthetic oligonucleotide”
attctctccttgccttcttta

SEQ ID NO: 45 - DNA, 21 bp, Artificial Sequence
  source: /note=“Description of Artificial Sequence Synthetic oligonucleotide”
agaatgtatgaagaacagtca

SEQ ID NO: 46 - DNA, 21 bp, Artificial Sequence
  source: /note=“Description of Artificial Sequence Synthetic oligonucleotide”
actctttaaagaaggcaagga

SEQ ID NO: 47 - DNA, 21 bp, Artificial Sequence
  source: /note=“Description of Artificial Sequence Synthetic oligonucleotide”
taagactctttaaagaaggca

SEQ ID NO: 48 - DNA, 21 bp, Artificial Sequence
  source: /note=“Description of Artificial Sequence Synthetic oligonucleotide”
gcaaaatatcaagtatatatg

SEQ ID NO: 49 - DNA, 21 bp, Artificial Sequence
  source: /note=“Description of Artificial Sequence Synthetic oligonucleotide”
aaaatctacattgatagcctt

SEQ ID NO: 50 - DNA, 21 bp, Artificial Sequence
  source: /note=“Description of Artificial Sequence Synthetic oligonucleotide”
aacgggcaataagactcttta

SEQ ID NO: 51 - DNA, 21 bp, Artificial Sequence
  source: /note=“Description of Artificial Sequence Synthetic oligonucleotide”
tatcagcatttacgacttata

SEQ ID NO: 52 - DNA, 21 bp, Artificial Sequence
  source: /note=“Description of Artificial Sequence Synthetic oligonucleotide”
atatggtgggctatttttcaa

SEQ ID NO: 53 - DNA, 21 bp, Artificial Sequence
  source: /note=“Description of Artificial Sequence Synthetic oligonucleotide”
agactctttaaagaaggcaag

SEQ ID NO: 54 - DNA, 21 bp, Artificial Sequence
  source: /note=“Description of Artificial Sequence Synthetic oligonucleotide”
tgccaaaatcccaaacttcag

SEQ ID NO: 55 - DNA, 21 bp, Artificial Sequence
  source: /note=“Description of Artificial Sequence Synthetic oligonucleotide”
attttaaagtactcattctca

SEQ ID NO: 56 - DNA, 21 bp, Artificial Sequence
  source: /note=“Description of Artificial Sequence Synthetic oligonucleotide”
ttgatttctgccagcatgtga

SEQ ID NO: 57 - DNA, 21 bp, Artificial Sequence
  source: /note=“Description of Artificial Sequence Synthetic oligonucleotide”
gtgatacgaaagttatctccc

SEQ ID NO: 58 - DNA, 21 bp, Artificial Sequence
  source: /note=“Description of Artificial Sequence Synthetic oligonucleotide”
tatctccctggaattgaccat

SEQ ID NO: 59 - DNA, 21 bp, Artificial Sequence
  source: /note=“Description of Artificial Sequence Synthetic oligonucleotide”
ctgaagataagggacagtttt

SEQ ID NO: 60 - DNA, 21 bp, Artificial Sequence
  source: /note=“Description of Artificial Sequence Synthetic oligonucleotide”
tgtgatacgaaagttatctcc

SEQ ID NO: 61 - DNA, 21 bp, Artificial Sequence
  source: /note=“Description of Artificial Sequence Synthetic oligonucleotide”
ttgtctccaaaaaactttctc

SEQ ID NO: 62 - DNA, 21 bp, Artificial Sequence
  source: /note=“Description of Artificial Sequence Synthetic oligonucleotide”
gtatcacaaaagcagatgctg

SEQ ID NO: 63 - DNA, 21 bp, Artificial Sequence
  source: /note=“Description of Artificial Sequence Synthetic oligonucleotide”
atctttcccaatggtcaattc

SEQ ID NO: 64 - DNA, 21 bp, Artificial Sequence
  source: /note=“Description of Artificial Sequence Synthetic oligonucleotide”
ttatctccctggaattgacca

SEQ ID NO: 65 - DNA, 21 bp, Artificial Sequence
  source: /note=“Description of Artificial Sequence Synthetic oligonucleotide”
ctttcccaatggtcaattcca

SEQ ID NO: 66 - DNA, 21 bp, Artificial Sequence
  source: /note=“Description of Artificial Sequence Synthetic oligonucleotide”
atctccctggaattgaccatt

SEQ ID NO: 67 - DNA, 21 bp, Artificial Sequence
  source: /note=“Description of Artificial Sequence Synthetic oligonucleotide”
gtccaccagattagctctggc

SEQ ID NO: 68 - DNA, 21 bp, Artificial Sequence
  source: /note=“Description of Artificial Sequence Synthetic oligonucleotide”
tccaccagattagctctggcc

SEQ ID NO: 69 - DNA, 21 bp, Artificial Sequence
  source: /note=“Description of Artificial Sequence Synthetic oligonucleotide”
tggacctcatgaaggcactgt

SEQ ID NO: 70 - DNA, 21 bp, Artificial Sequence
  source: /note=“Description of Artificial Sequence Synthetic oligonucleotide”
agagctaatctggtggacctc

SEQ ID NO: 71 - DNA, 21 bp, Artificial Sequence
  source: /note=“Description of Artificial Sequence Synthetic oligonucleotide”
ttagagccattgcttccaggc

SEQ ID NO: 72 - DNA, 21 bp, Artificial Sequence
  source: /note=“Description of Artificial Sequence Synthetic oligonucleotide”
aagtattcagacagtgccttc

SEQ ID NO: 73 - DNA, 36 bp, Artificial Sequence
  source: /note=“Description of Artificial Sequence Synthetic oligonucleotide”
gaatctaagtacggaccgccctgccccccttgccct

SEQ ID NO: 74 - protein, 12 aa, Artificial Sequence
  source: /note=“Description of Artificial Sequence Synthetic peptide”
ESKYGPPCPPCP

SEQ ID NO: 75 - protein, 119 aa, Artificial Sequence
  source: /note=“Description of Artificial Sequence Synthetic polypeptide”
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 229 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 282 aa, Unknown
  source: /note=“Description of Unknown IgD Spacer Sequence”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 78 - protein, 24 aa, Artificial Sequence
  source: /note=“Description of Artificial Sequence Synthetic peptide”
LEGGGEGRGSLLTCGDVEENPGPR

SEQ ID NO: 79 - protein, 357 aa, Artificial Sequence
  source: /note=“Description of Artificial Sequence Synthetic polypeptide”
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 80 - protein, 27 aa, Homo sapiens
FWVLVVVGGVLACYSLLVTVAFIIFWV

SEQ ID NO: 81 - protein, 66 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWV

SEQ ID NO: 82 - protein, 41 aa, Homo sapiens
RSKRSRLLHSDYMNMTPRRPGPTRKHYQPYAPPRDFAAYRS

SEQ ID NO: 83 - protein, 41 aa, Artificial Sequence
  source: /note=“Description of Artificial Sequence Synthetic polypeptide”
RSKRSRGGHSDYMNMTPRRPGPTRKHYQPYAPPRDFAAYRS

SEQ ID NO: 84 - protein, 42 aa, Homo sapiens
KRGRKKLLYIFKQPFMRPVQTTQEEDGCSCRFPEEEEGGCEL

SEQ ID NO: 85 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 112 aa, Artificial Sequence
  source: /note=“Description of Artificial Sequence Synthetic polypeptide”
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 112 aa, Artificial Sequence
  source: /note=“Description of Artificial Sequence Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - DNA, 20 bp, Unknown
  source: /note=“Description of Unknown CBLB Target Sequence”
aagcaagctgccgcagatcg

SEQ ID NO: 89 - DNA, 20 bp, Unknown
  source: /note=“Description of Unknown CBLB Target Sequence”
taagcaagctgccgcagatc

SEQ ID NO: 90 - DNA, 20 bp, Unknown
  source: /note=“Description of Unknown CBLB Target Sequence”
ctaagcaagctgccgcagat

SEQ ID NO: 91 - DNA, 20 bp, Unknown
  source: /note=“Description of Unknown CBLB Target Sequence”
tggaattgaccattgggaaa

SEQ ID NO: 92 - DNA, 20 bp, Unknown
  source: /note=“Description of Unknown CBLB Target Sequence”
tcatgaggtccaccagatta

SEQ ID NO: 93 - protein, 30 aa, Artificial Sequence
  source: /note=“Description of Artificial Sequence Synthetic polypeptide”
PGGGSGGGGSGGGGSGGGGSGGGGSGGGGP

SEQ ID NO: 94 - protein, 35 aa, Artificial Sequence
  source: /note=“Description of Artificial Sequence Synthetic polypeptide”
PGGGSGGGGSGGGGSGGGGSGGGGSGGGGSGGGGP

SEQ ID NO: 95 - protein, 17 aa, Artificial Sequence
  source: /note=“Description of Artificial Sequence Synthetic peptide”
GSADDAKKDAAKKDGKS

SEQ ID NO: 96 - DNA, 10 bp, Artificial Sequence
  source: /note=“Description of Artificial Sequence Synthetic oligonucleotide”
  misc_feature (1)..(10): /note=“This sequence may encompass 1-10 nucleotides”
aaaaaaaaaa

SEQ ID NO: 97 - DNA, 20 bp, Artificial Sequence
  source: /note=“Description of Artificial Sequence Synthetic oligonucleotide”
  misc_feature (1)..(20): /note=“This sequence may encompass 10-20 nucleotides”
aaaaaaaaaaaaaaaaaaaa

SEQ ID NO: 98 - DNA, 200 bp, Artificial Sequence
  source: /note=“Description of Artificial Sequence Synthetic polynucleotide”
  misc_feature (1)..(200): /note=“This sequence may encompass 25-200 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa